(19) United States
(12) Patent Application Publication
(10) Pub. No.: US 2012/0164073 A1
(43) Pub. Date: Jun. 28, 2012

(54) Stable nanoparticles, nanoparticle-based imaging systems, nanoparticle-based assays, and in vivo assays for screening biocompatibility and toxicity of nanoparticles

(75) Inventors: Xiaohong Nancy Xu, Norfolk, VA (US); Prakash Nallathamby, Norfolk, VA (US); Kerry J. Lee, Virginia Beach, VA (US)

(73) Assignee: Old Dominion University

(21) Appl. No.: 12/219,223

(22) Filed: Jul. 17, 2008

Related U.S. Application Data
(60) Provisional application No. 60/991,548, filed on Nov. 30, 2007.
(60) Provisional application No. 61/059,639, filed on Jun. 6, 2008.

(51) Int. Cl. A61K 49/00; G01N 21/47; G01N 21/49; B22F 9/24; B82Y 5/00; B82Y 40/00; B82Y 30/00

(52) U.S. Cl. 424/9.2; 424/9.1; 435/29; 356/337; 75/370; 977/840; 977/773; 977/810; 977/904; 977/896; 977/881

(57) ABSTRACT

Nanoparticles, such as noble metal nanoparticles, having improved stability against aggregation in aqueous solution are provided. In addition to having improved stability against aggregation, the nanoparticles are highly photostable. Also provided are aqueous solutions containing the stabilized nanoparticles, methods of making the stabilized nanoparticles, imaging methods and organisms employing the nanoparticles, and in vivo assays for screening and characterizing the toxicity and biocompatibility of the nanoparticles.

### RELATED APPLICATIONS

This application claims priority to U.S. provisional Ser. No. 60/991,548 filed Nov. 30, 2007, and also to U.S. provisional Ser. No. 61/059,639 filed Jun. 6, 2008, both of which are hereby incorporated by reference in their entireties.

### FEDERAL FUNDING STATEMENT

Various embodiments described herein were funded by the federal government under the following grants: NSF (NIRT BES 0507036), NIH (R01 GM076440), and NIH (RR15057-01). The government has certain rights in the invention.

### BACKGROUND

The production of nanoparticles, including noble metal nanoparticles, that do not aggregate in aqueous solution or photodecompose has proven quite challenging and has limited the number of applications in which such nanoparticles can be utilized. If nanoparticles are stable in solution only for days, they have to be resynthesized and recharacterized after they have been stored, used, or studied over longer periods of time. Such re-synthesis and re-characterization is both time-consuming and expensive, and is especially problematic for biological experiments that need days or weeks to be completed. Furthermore, in certain applications, it is important for nanoparticles to exhibit stability against aggregation for months, in order for them to become commercially viable and robust probes, and to be accessible to a wide spectrum of users. In fact, the poor stability and/or photodecomposition of nanoparticles has been a primary obstacle for achieving a wide variety of applications of nanoparticle probes.

Particle tracking has been used to investigate the dynamics of molecules and their surrounding media in vitro and in vivo. (See for example Apgar, J.; Tseng, Y.; Fedorov, E.; Herwig, M. B.; Almo, S. C.; Wirtz, D. Biophys. J. 2000, 79, 1095-1106; Daniels, B. R.; Masi, B. C.; Wirtz, D. Biophys. J. 2006, 90, 4712-4719; Jacobson, K.; Ishihara, A.; Inman, R. Annu. Rev. Physiol. 1987, 49, 163-175; Kusumi, A.; Nakada, C.; Ritchie, K.; Murase, K.; Suzuki, K.; Murakoshi, H.; Kasai, R. S.; Kondo, J.; Fujiwara, T. Annu. Rev. Biophys. Biomol. Struct. 2005, 34, 351-378; Kusumi, A.; Sako, Y. J. Cell Biology 1994, 125, 1251-1264; and Kusumi, A.; Sako, Y.; Yamamoto, M. Biophys. J. 1993, 65, 2021-2040.) However, many of the particles that have been used were relatively large, with diameters greater than 40 nm or even in the micrometer range. Such particles are heavy and hence diffuse slowly, offering inadequate sensitivity and limited temporal and spatial resolution for determining dynamic events of interest in vivo. Also, previously reported studies did not characterize the size distribution of the particles used therein, nor did they investigate the effect of particle size. Furthermore, the reported particle tracking studies have not provided methods to determine the sizes of particles in situ and in real-time, prohibiting the selection of identically sized nanoparticles to simultaneously investigate several dynamic events of interest in real-time.

Fluorescence probes have been used to study living biological system, but they suffer several drawbacks including photodecomposition, limiting the time available for probing dynamic events of interest.

The physical and surface properties specific to nanoparticles may also incite toxicity, damaging in vivo systems of interest or even posing risks to human health and the environment. Thus, a better understanding of the toxicity effect of the nanoparticles upon living organisms is desirable and the development of in vivo assays for screening the biocompatibility and/or toxicity of nanomaterials is in high demand.

### SUMMARY

The following abbreviations, used throughout this disclosure, are defined as follows:
DLS: dynamic light scattering;
hpf: hours-post-fertilization;
HRTEM: high resolution transmission electron microscopy;
LSPR: localized surface plasmon resonance;
LSPRS: localized surface plasmon resonance spectra;
RTSD: real-time square displacement;
SNOMS: dark-field single nanoparticle optical microscopy and spectroscopy; dark-field microscopy and spectroscopy;
ms: millisecond;
nm: nanometer.

Nanoparticles, such as noble metal nanoparticles, having improved stability against aggregation in aqueous solution are provided in at least some embodiments. In addition to having improved stability against aggregation in at least some embodiments, the nanoparticles also have a high degree of photostability in at least some embodiments (i.e., they are stable against photodecomposition and they are non-blinking). Also provided in at least some embodiments are aqueous solutions containing the stabilized nanoparticles and methods of making the stabilized nanoparticles. The stabilized nanoparticles in at least some embodiments can exhibit improved resistance to aggregation in aqueous solution, without the need for adding surface stabilizing agents. In at least some embodiments, the improved stability of the nanoparticles can be attributed to an increase in the absolute value of their surface zeta potentials, which can be achieved by treating the surfaces of the nanoparticles with an appropriate washing agent.

In at least some embodiments, the present methods for producing an aqueous solution of stabilized nanoparticles can comprise: (i) increasing the absolute value of the surface zeta potential of the nanoparticles; and (ii) dispersing the nanoparticles in an aqueous solution, wherein the nanoparticles exhibit improved stability against aggregation in the aqueous solution. Increasing the absolute value of the surface zeta potential can be accomplished by washing the nanoparticles with a washing agent that increases the thickness of the electrical double layer around the nanoparticles. Deionized water is an example of a suitable washing agent. The resulting aqueous solution comprises dispersed, non-aggregated nanoparticles, wherein the solution is substantially free of steric stabilizing agents and further wherein the nanoparticles are capably of remaining substantially non-aggregated in the aqueous solution for extended periods of time (e.g., at least one month or at least two months).

Methods of imaging nanoparticles in a biological organism in vivo and in real-time are also provided. These methods comprise exposing the biological organism to a plurality of nanoparticles, wherein the nanoparticles diffuse into the biological organism; and simultaneously imaging a plurality of individual nanoparticles within the biological organism in real-time by detecting light scattered by the nanoparticles, wherein the color of the scattered light is nanoparticle size-dependent. In one such method, the color of the light scattered by the nanoparticles can be imaged using SNOMS, and the color of the scattered light can be correlated to the size of the individual nanoparticles. By imaging the nanoparticles simultaneously and in real-time, information about various properties of the organism can be obtained. For example, the present imaging methods can be used to provide information about fluid viscosity, fluid flow, and transport dynamics in one or more environments within a biological organism in real-time.

Methods for determining the effect of nanoparticles on one or more living biological organisms in vitro or in vivo are also provided. These methods comprise exposing one or more living biological organisms to a plurality of nanoparticles and monitoring the effect of the nanoparticles on the morphology or development of the living biological organism. These methods may be used as biocompatibility and toxicity assays to determine the biocompatibility of the nanoparticles with one or more biological organisms by exposing the one or more biological organisms to multiple different concentrations of the nanoparticles, wherein exposure to at least one of the concentrations results in a physical abnormality in, or the death or, one or more of the biological organisms. Thus, by monitoring the nanoparticle concentration-dependent abnormality or death, the concentration-dependent biocompatibility of the nanoparticles can be determined. Similarly, the exposure time-dependent biocompatibility of the nanoparticles can be determined by exposing the biological organisms to the nanoparticles for multiple different exposure times.

Methods of transporting nanoparticles into an embryo are also provided. The method comprise exposing the embryo to a solution comprising a plurality of nanoparticles, wherein one or more nanoparticles passively diffuse into the embryo. The embryos can be transparent embryos, such as zebrafish embryos.

Although a variety of in vivo and in vitro systems and organisms can be used in, and benefit from, the present imaging methods and biocompatibility and toxicity assays, embryos are one non-limiting specific example. Zebrafish embryos can be a particularly useful biological organism because they are transparent, develop outside of their mothers, and undergo embryonic development rapidly with well-characterized developmental stages, which allows for the probing of events inside the embryos in vivo in real-time. More importantly, genetic screens of zebrafish phenotypes indicate similarities to human diseases, and protein sequences of drug-binding sites in zebrafish and human show a high degree of identity. Thus, zebrafish can serve as an effective in vivo model organism.

A variety of advantages can be found in various embodiments described herein including, for example:

In at least some embodiments, the nanoparticles can resist photodecomposition and blinking.

In at least some embodiments, very high or highest quantum yield (QY) can be achieved with single nanoparticle resolution.

In at least some embodiments, another advantage is multiple color probes for continuously imaging single living embryos and single living cells at nanometer spatial resolution in real-time for any desired time.

In at least some embodiments, expensive excitation sources are not needed.

In at least some embodiments, an inexpensive common halogen lamp can be used.

In at least some embodiments, another advantage is use of color index of nanoparticles as nanometer size index (CASI) for real-time imaging.

In at least some embodiments, another advantage is that nanoparticles can be easy to synthesize and can be purified with low cost.

Various descriptions can be found in Lee, K. J.; Nallathamby, P. D.; Browning, L. M.; Osgood, C. J.; Xu, X.-H. N. ACS Nano 1, 133-143 (2007); Nallathamby, P. D.; Lee, K. J., Xu, X.-H. N. ACS Nano Article ASAP, Web Release Date: 11-Jun.-2008 DOI: 10.1021/nn800048x (2008), which are herein incorporated by reference in their entireties including supporting information, including movies cited to therein and available to the public.

### BRIEF DESCRIPTION OF THE DRAWINGS

The patent or application file contains at least one drawing executed in color. Copies of this patent or patent application publication with color drawing(s) will be provided by the Office upon request and payment of necessary fee.

FIGS. 1A-1C show the characterization of the stability and optical properties of unwashed silver (Ag) nanoparticles: (a) immediately after synthesis and (b) 2 months after synthesis, respectively.

FIG. 1A shows UV-vis absorbance at 392 nm for a 1.04 nM solution of Ag nanoparticles of (a) 0.76 and (b) 0.69.

FIG. 1B shows histograms of the distribution of colors of single nanoparticles acquired by SNOMS: (a) (77±6)% blue, (21±6)% green and (2±1)% red and (b) (72±9)% blue, (23±6)% green and (5±3)% red.

FIG. 1C shows histograms of the size distribution of single nanoparticles measured by HRTEM as: (a) 11.6±2.4 nm and (b) 16.4±6.3 nm.

FIGS. 2A-2D show the characterization of the stability and optical properties of washed Ag nanoparticles: (a) immediately after synthesis and (b) 2 months after synthesis, respectively.

FIG. 2A shows the UV-vis absorbance at 392 nm of a 0.61 nM solution of Ag nanoparticles of (a) and (b) 0.59.

FIG. 2B shows HRTEM images of nearly uniform spherical nanoparticles. The scale bars are 10 nm.

FIG. 2C shows (i) a representative dark-field optical image of single nanoparticles and (ii) histograms of the distribution of colors of single nanoparticles acquired by SNOMS: (a) (74±6)% blue, (21±4)% green and (5±3)% red; and (b) (74±5)% blue, (20±4)% green and (6±1)% red.

FIG. 2D shows histograms of the size distribution of single nanoparticles at (i) (13.2±2.8) nm and (ii) (11.3±2.3) nm measured by (i) DLS and (ii) HRTEM, respectively. In (ii), the histogram also shows the correlation of colors with the sizes of individual nanoparticles: 78% nanoparticles are blue (8-18 nm), 16% nanoparticles are green (18-33 nm), and 5% nanoparticles are red (33-45 nm).

FIG. 3 shows: (A) a representative optical image of individual Ag nanoparticles with a uniform blue color; (B) a histogram showing that 99% of the nanoparticles of FIG. 3A were blue with 1% light blue and no occurrence of green and red. The dashed line and solid line squares illustrate the representative detection areas for measuring the integrated intensity of the background and presence of individual nanoparticles shown in FIGS. 4B-4C.

FIGS. 4A-4C show the characterization of the photostability of individual Ag nanoparticles.

FIG. 4A shows a representative LSPR spectrum (color) of a single Ag nanoparticle with a peak wavelength at 476 nm.

FIG. 4B shows representative plots of the scattering intensity from (i) a single nanoparticle and (ii) background versus illumination time.

FIG. 4C, zoom-in plots of FIG. 4B with a frame interval of 40.6 ms, show that single Ag nanoparticles resist photo-bleaching and blinking.

FIGS. 5A-5C illustrate real-time imaging and characterization of size-dependent diffusion of single Ag nanoparticles in solution.

FIG. 5A shows representative LSPR spectra of single Ag nanoparticles with peak wavelengths at 432 nm (i; blue nanoparticles), 543 nm (ii; green nanoparticles), and 655 nm (iii; red nanoparticles).

FIG. 5B shows the diffusion trajectories of the single nanoparticles of FIG. 5A in solution. Each pixel=0.067 μm.

FIG. 5C shows plots of RTSD of the single nanoparticles in FIG. 5B versus diffusion time; for the (i) blue, (ii) green, and (iii) red nanoparticles, respectively.

FIGS. 6A-6C illustrate real-time imaging and probing of the nano-environments of a living segmentation-stage (21 hpf) zebrafish embryo. Scale bar=100 μm in FIG. 6A and 10 μm in FIG. 6B.

FIG. 6A, an optical image of the living embryo, shows the inner mass of the embryo (IME), the yolk sac (YS), the chorion space (CS), and the chorion layers (CHL) of the embryo. The arrows show the counterclockwise flow patterns of embryonic fluid.

FIG. 6B, a zoom-in image of the CS region (marked by a square) in FIG. 6A, shows a close-up view of the CS and the diffusion trajectories of three single green nanoparticles (a) at the interface of the IME and the CS, (b) at the CHL, and (c) in the center of the CS. These images show distinctive diffusion patterns, indicating the different nano-environments of the embryo. The arrows show the flow direction of nanoparticles and flow patterns of embryonic fluid. Each pixel=0.067 μm.

FIG. 6C, plots of RTSD of the single green nanoparticles of FIG. 6B versus diffusion time, show distinctive diffusion modes.

FIGS. 7A-7F show characterization of the optical properties and stability of silver (Ag) nanoparticles.

FIG. 7A shows representative UV-vis absorption at 396 nm for a solution of 0.71 nM Ag nanoparticles in egg water at 28° C.: (a) immediately after synthesis (after 0 hours) and (b) 120 hours after synthesis (after 120 h), respectively; the peak absorbance and wavelength at 396 nm remained unchanged for 120 h.

FIG. 7B shows representative HRTEM images of nearly uniform spherical nanoparticles. Scale bar=5 nm.

FIG. 7C shows a representative dark-field optical image of single nanoparticles, showing that the majority of nanoparticles are blue, with some green and a few red.

FIG. 7D shows representative LSPR spectra (color) of single Ag nanoparticles with peak wavelengths at 452 nm (blue), 531 nm (green), and 601 nm (red).

FIG. 7E shows a histogram of the size and color distribution of individual Ag nanoparticles (average size: 11.6±3.5 nm): 74% nanoparticles are blue (5-15 nm), 23% nanoparticles are green (16-30 nm), and 1% nanoparticles are red (31-46 nm).

FIG. 7F shows representative plots of scattering intensity of (i) a representative single nanoparticle and (ii) background versus illumination time, showing that single Ag nanoparticles resist photobleaching and blinking.

FIGS. 8A-8F show optical images of a representative normal developmental-stage zebrafish in egg water in the absence of nanoparticles. Scale bar=500 μm. hpf=hours post-fertilization.

FIG. 8A shows a (1.25-1.50 hpf) 8-cell-stage embryo.

FIG. 8B shows a (2-2.25 hpf) 64-cell-stage embryo.

FIG. 8C shows a (24 hpf) segmentation-stage embryo.

FIG. 8D shows a (48 hpf) hatching-stage embryo.

FIG. 8E shows a (72 hpf) larval-stage embryo.

FIG. 8F shows a (120 hpf) completely developed zebrafish.

FIGS. 9A-9C illustrate real-time monitoring and characterizing of the transport of individual Ag nanoparticles in a living (2-2.25 hpf) 64-cell cleavage-stage embryo. LSPR spectra (color) of individual nanoparticles were used to distinguish them from tissue debris or vesicles in embryos. The time interval between sequential images in FIG. 9B and FIG. 9C is 25 s. Scale bar=400 μm in FIG. 9A and 15 μm in FIGS. 9B and 9C.

FIG. 9A, an optical image of a cleavage-stage embryo, shows chorionic space (CS), yolk sac (YS), and inner mass of the embryo (IME). The transport of single Ag nanoparticles at the interface of egg water with chorionic space, that of chorionic space with inner mass of the embryo, and inside chorionic space, are marked by B, C, and D, respectfully.

FIG. 9B shows sequential dark-field optical images, which illustrate the transport of single Ag nanoparticles (marked by a circle) from the egg water (extra-embryo) into the chorionic space via an array of chorion pore canals (CPCs) (marked by a rectangle).

FIG. 9C shows sequential dark-field optical images, which illustrate the transport of single Ag nanoparticles (marked by a circle) from chorionic space into inner mass of the embryo. The straight and curved dashed lines show the interfaces of inner mass of the embryo with chorionic space and that of chorionic space with egg water, respectively.

FIGS. 10A-10B show characterization of transport and diffusion of single Ag nanoparticles in a living cleavage-stage (64-cell) embryo.

FIG. 10A shows the diffusion trajectories of single Ag nanoparticles: (i) a red nanoparticle inside the chorion layers, (ii) a blue nanoparticle inside chorionic space, and (iii) a green nanoparticle at the interface of inner mass of the embryo and chorionic space.

FIG. 10B shows plots of RTSD versus diffusion time for the nanoparticles in FIG. 10A (a) in the living embryo: (i) a red nanoparticle in a restricted and stationary diffusion mode inside chorion pore canals with a diffusion coefficient D<1.9×10⁻¹¹ cm²; (ii) a blue nanoparticle in chorionic space away from the inner mass of the embryo with D=3.4×10⁻⁹ cm²/s ; and (iii) a green nanoparticle inside chorionic space near the surface of inner mass of the embryo with D=2.6×10⁻⁹ cm²/s; the nanoparticles in (ii) and (iii) both move in a simple Brownian motion; (b) in egg water for the single (i) blue, (ii) green, and (iii) red nanoparticles; all three types of nanoparticles show simple Brownian diffusion with D=8.4×10⁻⁸, 6.0×10⁻⁸, and 5.5×10⁻⁸ cm²/s, respectively.

FIGS. 11A-11B show characterization of Ag nanoparticles embedded in embryos using dark-field SNOMS.

FIG. 11A shows representative (a) color image and (b) LSPR spectra of single Ag nanoparticles embedded in the chorion layers; the nanoparticles inside the chorion layers have multiple colors (blue, green, red), and some of the nanoparticles are overlapped with the chorion pore canals (note an array of chorion pore canals is marked by a triangle). Scale bar=1 μm.

FIG. 11B shows representative images of individual Ag nanoparticles embedded in the chorion layers, illustrating that some nanoparticles (marked by a circle) were trapped in the chorion pore canals (marked by ellipses); (b) is a zoom-in of the CCD camera image shown in (a), and a zoom-in image of (b) is shown in (c), indicating that the dark-red nanoparticles clog the chorion pore canals. Scale bar=10 μm (a) and 2 μm (b), respectively. OC=outside chorion.

FIGS. 12A-12B show characterization of individual Ag nanoparticles embedded inside a fully developed (120 hpf) zebrafish using dark-field SNOMS. Scale bar=400 μm (A) and 4 μm (B).

FIG. 12A shows an optical image of a fixed, normally developed zebrafish, with representative areas marked by rectangles: (i) retina, (ii) brain (mesencephalon cavity), (iii) heart, (iv) gill arches, and (v) tail.

FIG. 12B shows a zoom-in optical images of those nanoparticles in FIG. 12A; the individual nanoparticles are marked by dashed circles.

FIGS. 13A-13D show histograms of distribution of normally developed (green) and dead (red) zebrafish.

FIG. 13A shows the percentage of normally developed (green) and dead (red) zebrafish versus concentration of Ag nanoparticles that the zebrafish embryos have been exposed to for 120 hours.

FIG. 13B, results from negative control, shows the percentage of normally developed (green) and dead (red) zebrafish versus concentration of supernatants collected from washing of the nanoparticles that the zebrafish embryos have been exposed to for 120 hours.

FIG. 13C shows a histogram of distribution of deformed zebrafish (120 hpf) versus concentration of the nanoparticles that the zebrafish embryos have been exposed to for 120 hours.

FIG. 13D shows a histogram of distribution of five representative types of deformities of the zebrafish versus concentration of the nanoparticles that the zebrafish embryos have been exposed to for 120 hours: finfold abnormality (purple), tail and spinal cord flexure and truncation (black), cardiac malformation (pink), yolk sac edema (light green), head edema (brown), and eye abnormality (orange).

FIGS. 14A-14G, representative optical images of zebrafish, show zebrafish that are normally developed (blank control) (FIG. 14A) and zebrafish with different abnormalities (FIG. 14B-14G. Scale bar=500 μm.

FIG. 14A shows normal development of (i) finfold, (ii) tail/spinal cord, (iii) cardiac, (iii, iv) yolk sac, cardiac, head, and eye.

FIG. 14B shows finfold abnormality.

FIG. 14C shows tail and spinal cord flexure and truncation.

FIG. 14D shows cardiac malformation.

FIG. 14E shows yolk sac edema.

FIG. 14F shows head edema, showing both (i) head edema and (ii) head edema and eye abnormality.

FIG. 14G shows eye abnormality, showing both (i) eye abnormality and (ii) eyeless.

### DETAILED DESCRIPTION

All publications, patent applications, and patents mentioned herein are incorporated by reference in their entirety including figures, claims, working examples, and supporting information.

### Synthesis of Stable Nanoparticles:

Nanoparticles having improved stability against aggregation in aqueous solution are provided. The solution can be a suspension of particles. Also provided are aqueous solutions containing the stabilized nanoparticles and methods of making the stabilized nanoparticles. The stabilized nanoparticles can exhibit improved resistance to aggregation (i.e., increased stability) in aqueous solution, without the need for adding surface stabilizing agents. The improved stability of the nanoparticles can be attributed to an increase in the absolute value of their surface zeta potentials, which can be achieved by washing the surfaces of the nanoparticles with an appropriate washing agent. A washing agent can be any aqueous solution, such as nanopure water. A detailed description of the synthesis of stable nanoparticles can be found in for example Nallathamby, P. D.; Lee, K. J., Xu, X.-H. N. ACS Nano Article ASAP, Web Release Date: 11-Jun.-2008 DOI: 10.1021/nn800048x (2008).

The present methods provide an aqueous solution comprising dispersed, non-aggregated nanoparticles and is substantially free of additional steric stabilizing agents, such as surface ligands or surfactants that have been used to help stabilize nanoparticles against aggregation.

The nanoparticles can be produced by adding at least one metal-containing compound, such as a silver-containing compound, into an aqueous medium, such as an aqueous solution. The medium can comprise at least two reducing agents that are dissolved in a medium such as water at a temperature of no greater than about 5° C., such as at about 0° C., while the solution is constantly being stirred. In one embodiment, after the addition of the reducing agents, the solution is further continuously stirred for a period of at least 8 hours, such as 12 hours. The reducing agents can be the same or different compounds, and they can be present in any ratio to each other in the solution. For instance, one reducing agent can comprise a sodium salt such as sodium citrate, and another can comprise another salt such as sodium borohydride, and the ratio of the former to the latter present in the solution can be for example about 1:5 to about 1:20, such as about 1:9 to about 1:11. The reducing agents can act to reduce the silver-containing compound to form silver nanoparticles. The solution of nanoparticles produced can be further filtered through a filter that has a mesh size of no greater than 5 microns, such as no greater than 2 microns, and thereafter washed with a washing agent, such as water. Deionized, or “DI,” water is one example of a suitable washing agent.

In one basic form, the method for producing an aqueous solution of stabilized nanoparticles comprises increasing the absolute value of the surface zeta potential of the nanoparticles and dispersing the nanoparticles in an aqueous solution, wherein the nanoparticles exhibit improved stability against aggregation in the aqueous solution. In some embodiments, the absolute value of the surface zeta potential may be increased by at least about 30%, about 50%, or at least about 70%. This includes embodiments wherein the absolute value of the surface zeta potential is increased by at least about 75%.

The stable nanoparticles are able to remain non-aggregated, or substantially non-aggregated, in aqueous solution for extended periods of time. This includes embodiments wherein the nanoparticles can remain substantially non-aggregated for a period of at least two weeks, at least one month, at least two months, or at least three months. As used herein, the phrase “substantially non-aggregated” includes collections of nanoparticles wherein the vast majority (e.g., 90% or 95%) remain non-aggregated in solution and is meant to cover situations wherein a number of nanoparticles may be aggregated, but wherein this small degree of aggregation does not have a measurable effect on nanoparticle concentration (number of individual nanoparticles in solution), does not affect the optical and physical properties of the nanoparticle solution, and/or does not render the collection of nanoparticles unsuitable for single-nanoparticle-resolution imaging and assay applications, such as those described herein.

The composition and size of the nanoparticles may vary. However, for imaging applications the nanoparticles desirably exhibit size-dependent LSPR spectra and, therefore, desirably have diameters that are sufficiently small to render their LSPR spectra size-dependent. Examples of such nanoparticles include nanoparticles composed of, or comprising, noble metals, such as silver, gold, copper, and alloys thereof In some such embodiments the nanoparticles will have an average diameter of no greater than about 100 nm. This includes nanoparticles that have average diameters of no greater than about 50 nm, no greater than about 40 nm, no greater than about 20 nm, and no greater than about 15 nm.

By way of illustration only, in one embodiment, the stabilized nanoparticles can be noble metal nanoparticles that have been washed one or more times with a washing agent. Washing the nanoparticles can remove species from the solution, in which nanoparticles are present, resulting in decrease in the ionic strength of solution and an increase in the thickness of the nanoparticles' electrical double layers in solution, which leads to an enhanced surface zeta potentials and improved stability against aggregation.

An embodiment of a method for producing an aqueous solution of stabilized nanoparticles is a method of producing stabilized silver nanoparticles by washing the silver nanoparticles with deinoized water to provide silver nanoparticles having surface zeta potentials of, for example, −25 mV or lower (e.g., as −35 mV or lower, −60 mV or lower, −100 mV or lower, or −130 mV or lower) and dispersing the stabilized silver nanoparticles in an aqueous dispersion medium such as water.

The nanoparticles can be highly photostable. The photostability (e.g., resistance to photodecomposition and blinking) of the nanoparticles can be demonstrated by the fact that their optical properties remain substantially unchanged over extended periods of time (hours or even days). An optical property of a nanoparticle can be considered to have remained “substantially unchanged” as long as that optical property remains sufficiently constant over a sufficient period of time to render the nanoparticles suitable for real-time imaging and assay applications, such as those described herein.

One optical property of the nanoparticles that may be used as a gauge of their photostability is the resistance to photodecomposition, as measured by the unchanging (or substantially unchanging) nature of the shape, area and/or location of the peak wavelength(s) in the LSPR spectra of the nanoparticles over time. The relevant period of time can depend, on the duration of the experiments or assays in which the nanoparticles are to be used. For example, in some embodiments, the LSPR spectra for the nanoparticles can remain substantially unchanged for a period of at least about an hour, at least about 5 hours, at least about 12 hours, at least about 24 hours, or even at least about 100 hours.

Another optical property that may be used to gauge the photostability of the nanoparticles is their resistance to photobleaching (i.e., photodecomposition) or blinking. Again, the relevant period of time can depend, on the duration of the experiments or assays in which the nanoparticles are to be used. Thus, in some embodiments, the nanoparticles exhibit no (or no significant) photobleaching and/or photoblinking for a period of at least about one hour, at least about 5 hours, at least about 12 hours, at least about 24 hours, or even at least about 100 hours.

As illustrated in the working examples, the improved stability and optical properties of the nanoparticles can be measured using a variety of methods including, but not limited to, UV vis absorption measurements, SNOMS, HRTEM, and DLS.

Because they are photostable and resist aggregation, the nanoparticles are ideal candidates for a variety of detection and imaging applications and can be imaged for their toxicity and biocompatibility via in vivo assays. Examples of such applications are described in more detail below.

Imaging Biological Organisms with Nanoparticles:

The stable nanoparticles provided herein are well-suited for a variety of detection and imaging systems, including in vitro and in vivo imaging of nanoparticles in biological organisms. The nanoparticles can be tracked in and/or around a biological organism in real-time and used to probe dynamic events in and around the biological organism. Because individual nanoparticles can be imaged and tracked individually and simultaneously with single-nanoparticle resolution, imaging a plurality of the individual nanoparticles in real-time can provide information about multiple environments in and around the biological organism in real-time. Imaging and detection can be considered to be done in “real-time” if, for example, dynamic events are monitored continuously, or substantially continuously, as they occur, although there may be some minimal deviation from true real-time resulting from limitations on the temporal resolution of the equipment used to image or detect the nanoparticles. Generally, any imaging or detection that is performed with a temporal resolution of a millisecond or better (e.g., a microsecond or better) can be considered “real-time” imaging. Descriptions of using nanoparticles to image biological organisms can be found in for example Lee, K. J.; Nallathamby, P. D.; Browning, L. M.; Osgood, C. J.; Xu, X.-H. N. ACS Nano 1, 133-143 (2007); Nallathamby, P. D.; Lee, K. J., Xu, X.-H. N. ACS Nano Article ASAP, Web Release Date: 11-Jun.-2008 DOI: 10.1021/nn800048x (2008).

One embodiment of the methods of imaging nanoparticles in a biological organism in vivo, comprises exposing the biological organism to a plurality of nanoparticles, wherein the nanoparticles diffuse into the biological organism; and simultaneously imaging a plurality of individual nanoparticles within a biological organism in vivo and in real-time by detecting light scattered by the nanoparticles, wherein the color of the scattered light is nanoparticle size-dependent. A plurality of individual nanoparticles (desirably having a monodisperse or substantially monodisperse size/color distribution) can be imaged simultaneously and in real-time in accordance with the present imaging methods, making it possible to probe such properties as fluid viscosities, fluid dynamics, and/or transport dynamics and mechanisms, for multiple environments in and/or around the biological organism in vivo and in real-time.

In vivo and in vitro methods are known in the art. For example, nanoparticles can be imaged “in vivo” if the nanoparticles are within a living multicellular organism, such as an embryo. Nanoparticles can be imaged “in vitro” if the nanoparticles are in an artificial environment outside of a living multicellular organism, or in a unicellular organism, such as a bacterium. Nanoparticles can be considered within a biological organism if they are in, on, in the immediate environment of, or surrounding the biological organism. For example, the nanoparticles can be located in various extracellular regions within a biological systems, such as on the surface of, or at an interface within, a biological organism.

SNOMS is a technique that is well-suited for imaging and tracking the nanoparticles, as illustrated by the working examples below. SNOMS can be used to determine the color of the light being scattered from individual nanoparticles via direct visualization of the LSPRS of the nanoparticles, and the color of the light can then be correlated to the sizes of the individual nanoparticles. Thus, sizes of individual nanoparticles can be imaged at a nanometer resolution via dark-field optical microscopy (SNOMS). SNOMS can be used to observe individual nanoparticles that are dispersed in an aqueous medium or on a relatively dry substrate. For example, the individual nanoparticles can be at an interface between an aqueous medium and air or at an interface between the medium and the solid container.

A variety of in vivo and in vitro systems can be used in, and benefit from, the present methods of imaging biological organisms. Examples of such systems include, but are not limited to, vertebrate biological organisms, biological tissues, cells and embryos. If the nanoparticles to be imaged are located within the biological organism of interest, that organism is desirably at least partially transparent to the incident and scattered light used in SNOMS. Zebrafish embryos can be a particularly useful biological organism because they are transparent, develop outside of their mothers, and undergo embryonic development rapidly with well-characterized developmental stages. In addition, as shown in the working examples, below, nanoparticles are able to diffuse into living zebrafish embryos through their chorion pore canals which allows for the probing of events inside the embryos in vivo in real-time. The embryos can be in different early developmental stages, such as cleavage-stage and segmentation-stage, of zebrafish embryos.

Because of the photostability of the nanoparticles, the imaging of nanoparticles in biological organisms in vivo can be performed continuously for a long period of time; for example, the duration of imaging can be at least 0.5 hours. This includes periods of at least 1 hour, at least 24 hours, or at least 48 hours.

One advantage of using such nanoparticles in a biological organism is that, in addition to functioning as a monitoring probe, the nanoparticles can be used as a drug delivery device. In such instances, the nanoparticles can carry at least one pharmaceutical drug. The drug can be associated with the nanoparticles by methods known in the art such as functionalization.

Monitoring Biological Organisms with Nanoparticles:

Nanoparticles can also be used in assays for determining the effect of the nanoparticles on various organisms, particularly biological organisms in vivo or in vitro. In these assays, one or more biological organisms are exposed to a plurality of nanoparticles and the effect of the nanoparticles on the morphology or development of the biological organism is monitored. One such method provides a biological assay for determining the biocompatibility and toxicity of the nanoparticles to biological organisms. Zebrafish embryos are well-suited as the biological organisms in the present in vivo assays for screening biocompatibility of nanomaterials because they develop quickly with well-defined developmental stages. In addition, these embryos develop outside of their mothers and are transparent, which facilitates the visual observation of their development. More importantly, genetic screens of zebrafish phenotypes indicate similarities to human diseases, and protein sequences of drug-binding sites in zebrafish and human show a high degree of identities. Descriptions of using nanoparticles to monitor biological systems can be found in for example Lee, K. J.; Nallathamby, P. D.; Browning, L. M.; Osgood, C. J.; Xu, X.-H. N. ACS Nano 1, 133-143 (2007); Nallathamby, P. D.; Lee, K. J., Xu, X.-H. N. ACS Nano Article ASAP, Web Release Date: 11-Jun.-2008 DOI: 10.1021/nn800048x (2008).

Other types of biological systems could also be used. For example, various fish embryos or vertebrate model system (e.g., frog) that share similar traits with zebrafish can be studied using the present imaging systems, and in vivo assay for screening biocompatibilities of nanomaterials.

The biocompatibility and toxicity of nanoparticles can be determined as a function of nanoparticle concentration, nanoparticle exposure time, or both by using an in vivo assay. Responses of the biological organism, such as a zebrafish embryo, can depend on the concentration of the nanoparticles to which it is exposed. For example, in one assay, one or more biological organisms (e.g., a plurality of zebrafish embryos) are exposed to nanoparticles at multiple different nanoparticle concentrations. The different concentrations can be span a range of concentrations, such that exposure to at least one of the concentrations results in a physical abnormality in, or the death of, one or more of the biological organisms. By monitoring the effects of the nanoparticles on the morphology and/or development of the organisms, the concentration-dependent biocompatibility of the nanoparticles can be determined. In another assay, one or more biological organisms (e.g., a plurality of zebrafish embryos) are exposed to nanoparticles for different exposure times. The different exposure times can be span a range of times, such that exposure for at least one of the exposure times results in a physical abnormality in, or the death of, one or more of the biological organisms. By monitoring the effects of the nanoparticles on the morphology and/or development of the organisms, the exposure time-dependent biocompatibility of the nanoparticles can be determined.

Imaging the nanoparticles in a biological organism that has developed a physical abnormality (e.g., a physical mutation) or that had died as a result of the exposure to the nanoparticles can provide information on the location, amount and/or sizes of the nanoparticles associated with the biological organism. This information can be correlated to the abnormality or death. As such, the present methods can be used to develop tools for the diagnosis and therapy of in vivo organisms.

Also provided herein are kits which can comprise one or more elements as described herein to carry out one or more applications as described herein. For example, a kit can comprise a collection of nanoparticles or a solution of nanoparticles in a container, instructions for use of the nanoparticles, and one or more other elements useful for a test or assay.

### NON-LIMITING WORKING EXAMPLES

### Example 1

### Methods for Forming Stable Silver Nanoparticles

This example demonstrates methods for producing silver nanoparticles that are photostable and resist aggregation.

### Reagents and Supplies:

Sodium borohydride, NaBH₄, (>98%, EMD), sodium citrate (Sigma-Aldrich) and AgClO₄ (>99.9%, Alfa Aesar), were purchased and used without further purification. Nanopure water (Nanopore, 18 MΩ) was used to prepare all solutions and rinse all glassware.

Synthesis of Ag nanoparticles:

Silver nanoparticles were synthesized by reducing AgClO₄ with NaBH₄ and sodium citrate as follows: sodium citrate dihydrate (0.3 mM) and NaBH₄ (10 mM) were dissolved in 247.5 mL ice-cold nanopure water. AgClO₄ (2.5 mL, 10 mM) was rapidly added to the reaction mixture and the solution was homogeneously stirred at 425 rpm overnight at room temperature. After the reaction was completed, the solution was immediately filtered using 0.2 μm sterilized membrane filters (Whatman).

To produce washed nanoparticles, the Ag nanoparticles were immediately washed twice with an aqueous solution using centrifugation. In one embodiment, the solution is nanopure water and centrifugation is performed at 15,000 rcf (relative centrifugal force). The washed Ag nanoparticles were resuspended in nanopure water.

Both washed and unwashed Ag nanoparticle solutions were stored in the dark at 4° C. until further characterization.

### Characterization of the Stability of Silver Nanoparticles:

The concentration, size and optical properties of unwashed and washed Ag nanoparticles were characterized over a period of two months after their synthesis to investigate their stability. The stability measurements were carried out using UV-Vis spectroscopy (Hitachi U-2010), SNOMS, HRTEM (FEI Tecnai G2 F30 FEG, at 300 kV), and DLS (Nicomp 380ZLS particle sizing system). Zeta potentials of the nanoparticles in solution were also measured using the Nicomp 380ZLS particle sizing system. Nanoparticle concentrations were calculated as described in Xu, X.-H. N.; Huang, S.; Brownlow, W.; Salatia, K.; Jeffers, R. J. Phys. Chem. E. 2004, 108, 15543-15551, and Huang, T.; Nallathamby, P. D.; Gillet, D.; Xu, X.-H. N., Anal. Chem. 2007, 79, 7708-7718.

A description of the SNOMS technique may be found in the following references: Xu, X.-H. N.; Song, Y.; Nallathamby, P. D., Probing membrane transport of single live cells using single molecule detection and single nanoparticle assay; New Frontiers in Ultrasensitive Bioanalysis: Advanced Analytical Chemistry Applications in Nanobiotechnology, Single Molecule Detection, and Single Cell Analysis, Xu, X.-H. N., Ed. Wiley: New Jersey, 2007; pp 41-65; Huang, T.; Nallathamby, P. D.; Gillet, D.; Xu, X.-H. N. Anal. Chem. 2007, 79, 7708-7718; Kyriacou, S.; Brownlow, W.; Xu, X.-H. N. Biochemistry 2004, 43, 140-147; Xu, X.-H. N.; Brownlow, W. J.; Kyriacou, S. V.; Wan, Q.; Viola, J. J. Biochemistry 2004, 43, 10400-10413; Xu, X.-H. N.; Chen, J.; Jeffers, R. B.; Kyriacou, S. V. Nano Letters 2002, 2, 175-182. In this example, an EMCCD (PhotonMAX) equipped with a spectrograph (SpectraPro-150) (Roper Scientific) was used for LSPR spectra characterization of single nanoparticles. An EMCCD, a high-resolution CCD camera (Micromax, 5 MHz Interlin) (Roper Scientific) and color digital camera (Nikon and Sony) were used for imaging and characterization of single nanoparticles in solution.

### Characterization of the Unstability of Unwashed Silver Nanoparticles:

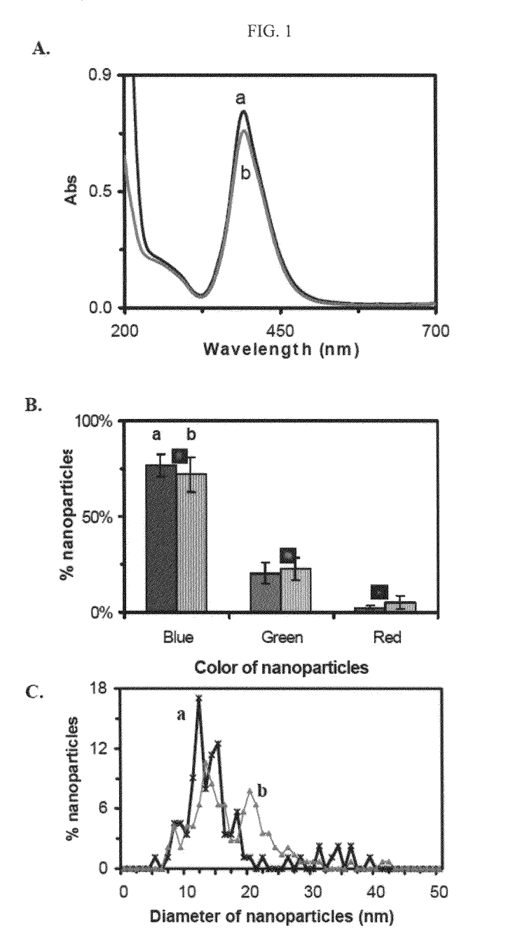

Stability measurements for the unwashed silver nanoparticles are shown in FIGS. 1A-C. FIG. 1A shows the UV-vis spectra of a 1.04 nM aqueous solution of unwashed silver nanoparticles measured at 392 nm: (a) immediately after and (b) 2 months after their synthesis. The UV-vis absorption spectrum of the Ag nanoparticles immediately after synthesis showed an absorbance of 0.76 with a peak wavelength at 392 nm and a peak width at half maximum (FWHM) of 63 nm, indicating a narrow size distribution. The UV-vis absorbance for the unwashed silver nanoparticles began to decrease within 24 hours after synthesis. By the end of 2 months, the peak absorbance decreased to 0.69 from an initial value of 0.76 with a slightly wider FWHM of 66 nm, as shown in FIG. 1A(b).

FIG. 1B shows histograms representing the LSPRs color distribution of the unwashed silver nanoparticles as measured by SNOMS. The results show that the color distribution of the nanoparticles in solution immediately after formation was as follows: 77% blue nanoparticles, 21% green and 2% red nanoparticles. In contrast, the histogram of the color distribution of the silver nanoparticles after 2 months (FIG. 1B(b)) showed 72% blue nanoparticles, 23% green and 5% red nanoparticles, suggesting more green and red nanoparticles than in the solution immediately after synthesis.

FIG. 1C shows histograms of the size distribution of the unwashed silver nanoparticles as measured by HRTEM. As shown in FIG. 1C(a), the average size of the silver nanoparticles immediately after formation was 10.7±2.4 nm and their shape was spherical. Histograms of the size distribution measured by HRTEM after two months (FIG. 1C(b)) illustrate an increase in size and distribution from 10.7±2.4 nm to 16.4±6.3 nm, suggesting that the nanoparticles were unstable and became less monodisperse over time. As the size of nanoparticles increased, nanoparticle concentration decreased, and their size-dependent surface and optical properties changed.

### Characterization of the Stability of Washed Silver Nanoparticles:

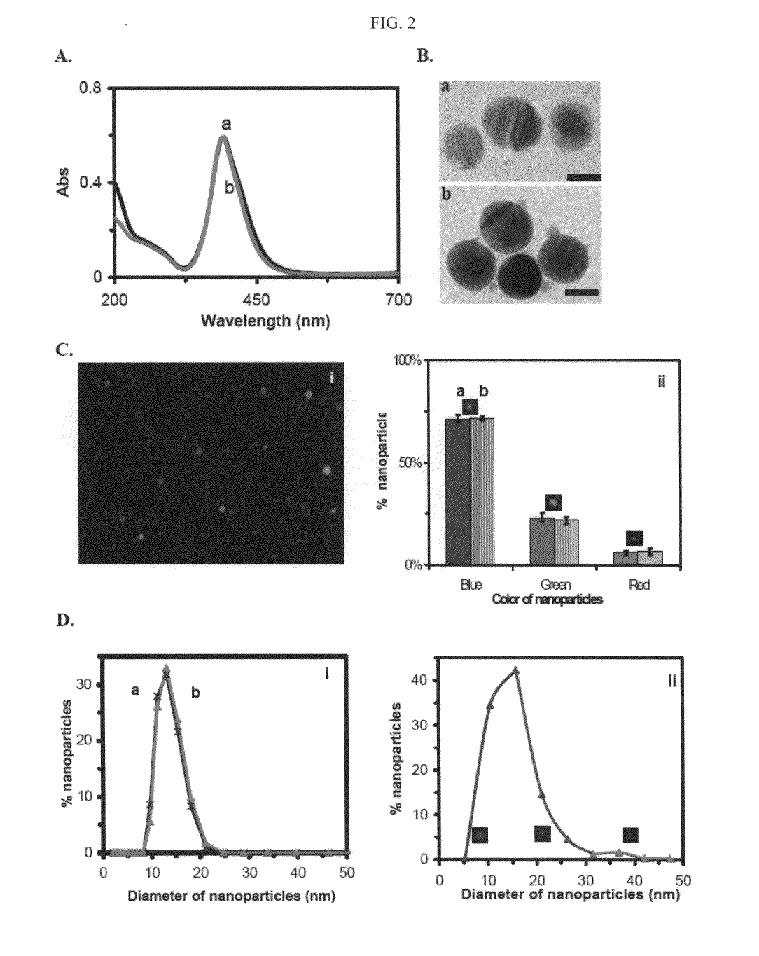

Stability measurements for the washed silver nanoparticles are shown in FIGS. 2A-D. The data show that the washed nanoparticles were much more stable than unwashed nanoparticles, and that their physical and optical properties remained nearly constant over a period of at least 2 months. The zeta potential (ζ) of the nanoparticles decreased from −19.96 mV to −35 mV after washing, showing an increase in surface charge and thereby enhanced stability in solution. Without intending to be bound to any theory, it is believed that removing chemicals from the surface of the nanoparticles during washing led to a decreased ionic strength solution, which increased the thickness of electrical double layer and hence enhanced the zeta potential and surface charge of the nanoparticles.

FIG. 2A shows the UV-vis spectra of a 0.61 nM aqueous solution of silver nanoparticles measured at 392 nm (a) immediately after washing and (b) two months after washing. As shown in FIG. 2A, the UV-vis spectra of the washed nanoparticles showed an absorbance of 0.59 with a peak wavelength at 392 nm and FWHM of 62 nm, which remained constant over 2 months.

FIG. 2B shows HRTEM images of the nanoparticles. These HRTEM images show that the spherical shape of the washed nanoparticles. The histograms of the size distribution of the nanoparticles in solution, determined using DLS and HRTEM, showed that the average size of the nanoparticles was 13.2±2.8 nm and 11.3±2.3 nm, respectively, and that the size and spherical shape of the nanoparticles remained unchanged over 2 months. Hydrodynamic radii of nanoparticles in solution measured by DLS are always larger than those for dried nanoparticles measured using TEM because nanoparticles are hydrated in solution. That is why slightly larger radii are observed for nanoparticles using DLS than using HRTEM.

FIG. 2C shows a representative dark field optical image of individual nanoparticles and histograms representing the color distribution of the washed silver nanoparticles, as acquired by SNOMS. The histograms, which remain substantially unchanged over two months, show that the majority (74%) of nanoparticles were blue with 21% green and 5% red.

Histograms showing the size distribution and SNOMS measurements showing the color (LSPRS) distribution of the single silver nanoparticles showed a correlation of color with size, indicating that 74% of the nanoparticles had diameters of 5-15 nm, 20% of the nanoparticles had diameters of 16-33 nm, and 6% of the nanoparticles had diameters of 34-45 nm (see FIG. 2D). These size ranges correlated with blue, green and red nanoparticles, respectively. Thus, the color index of Ag nanoparticles can be correlated to the size index. This approach allows for the effective use of the color (LSPRS) of individual nanoparticles to determine their sizes in solution at nanometer resolution using SNOMS. Notably, despite numerous studies, (see, for example, Bohren, C. F.; Huffman, D. R. Absorption and Scattering of Light by Small Particles; Wiley: New York, 1983; pp. 287-380, and references therein; Kreibig U. Vollme M. Optical Properties of Metal Clusters; Springer: Berlin, 1995; pp. 14-123; and Mie, G. Ann. Phys. 1908, 25, 377-445) currently it is still impossible to accurately compute and describe the LSPRS of individual nanoparticles prepared by chemical synthesis because it appears that the surface morphologies and adsorbates of nanoparticles and surrounding medium play an important role in the optical properties of nanoparticles. (Wiley, B.; Sun, Y.; Xia, Y. Acc. Chem. Res. 2007, 40, 1067-1076, and references therein.) The present experimental approach of correlating size distribution with color (LSPRS) distribution is based on statistics and calibration approaches, which accurately reflects the size and color distribution of the nanoparticles in a particular nanoparticle solution at single nanoparticle resolution.

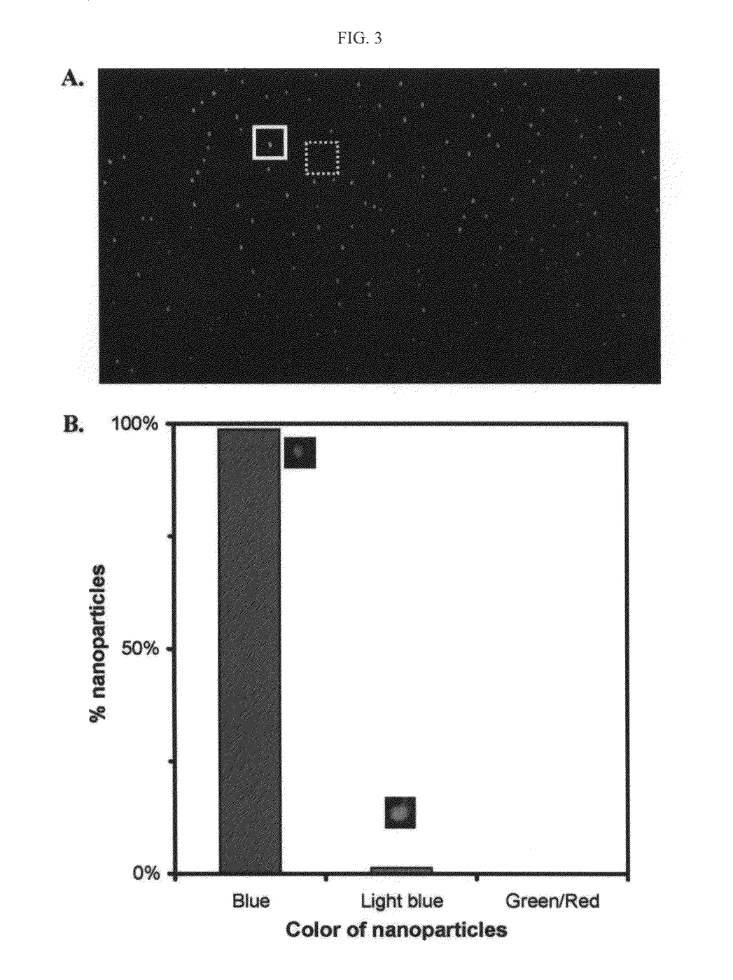

The synthesis and washing conditions can be tailored to provide monodisperse, or substantially monodisperse, silver nanoparticles. FIG. 3A and 3B show a representative optical image of individual silver nanoparticles showing uniform blue color and a histogram of such a monodisperse collection of silver nanoparticles, wherein all nanoparticles are blue (99% blue and 1% light blue), respectively. Blue nanoparticles are smaller nanoparticles (5-15 nm). Thus, they have lower quantum yield (QY) of Rayleigh scattering and are less sensitively detected than green and red nanoparticles. Nevertheless, blue nanoparticles can be directly observed and characterized using SNOMS.

### Characterization of the Photostability of Silver Nanoparticles:

The photostability (i.e., resistance to photodecomposition and blinking) of the nanoparticles was determined by acquiring sequential optical images of single Ag nanoparticles using an EMCCD camera with exposure time at 100 ms and readout time of 40.6 ms while these nanoparticles were constantly radiated under a dark-field microscope illuminator (100 W halogen) for 12 hr. The illumination power at the sample stage (focal plane of dark field) was 0.070±0.001 watts during the experiment. The scattering intensity of individual nanoparticles within a 20×20 pixel area was measured and the average background intensity of several detection areas with the same size (20×20 pixels) was also acquired in the absence of nanoparticles. The average background intensity was subtracted from the integrated intensity and the background, and the subtracted integrated intensity was plotted as a function of time. The fluctuations in the intensity from the nanoparticles were compared with those of the background to determine the photostability of the silver nanoparticles.

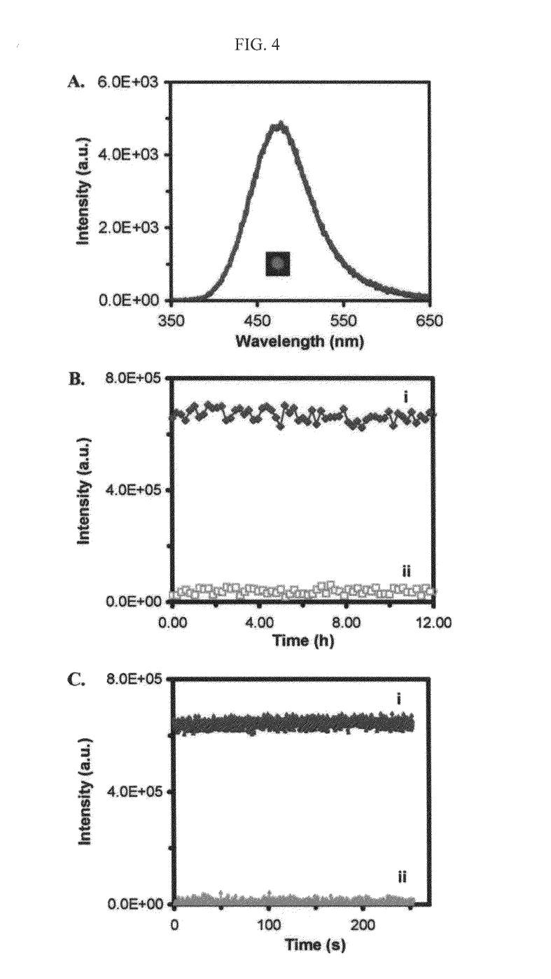

The LSPR spectra of single nanoparticles before and after 12 hours of illumination showed that the spectra remained unchanged (FIG. 4A). Plots of scattering intensity of single nanoparticles and background (in the absence of nanoparticles) versus illumination time (shown in FIG. 4B) show that the scattering intensity of individual single nanoparticles remained unchanged over 12 h. The zoom-in plots of FIG. 4B with a short frame interval of 40.6 ms further illustrate that small fluctuations in the scattering intensity of the single nanoparticles (FIGS. 4B and 4C: i) were similar to the intensity fluctuations of the background (FIGS. 4B and 4C: ii), showing that the intensity fluctuations were attributable to the illuminator and the noise level of the CCD camera. Thus, the results shown in FIG. 4 demonstrate that the single Ag nanoparticles resist photodecomposition and blinking.

### Example 2

### Using Single Nanoparticle Optics to Image and Assay Zebrafish Embryos in Real-Time

This example illustrates the use of silver nanoparticles, which are made in accordance with the methods of Example 1, and an imaging system for probing zebrafish embryos in vivo in real-time.

### Characterization of the Size-Dependent Diffusion of Single Nanoparticles:

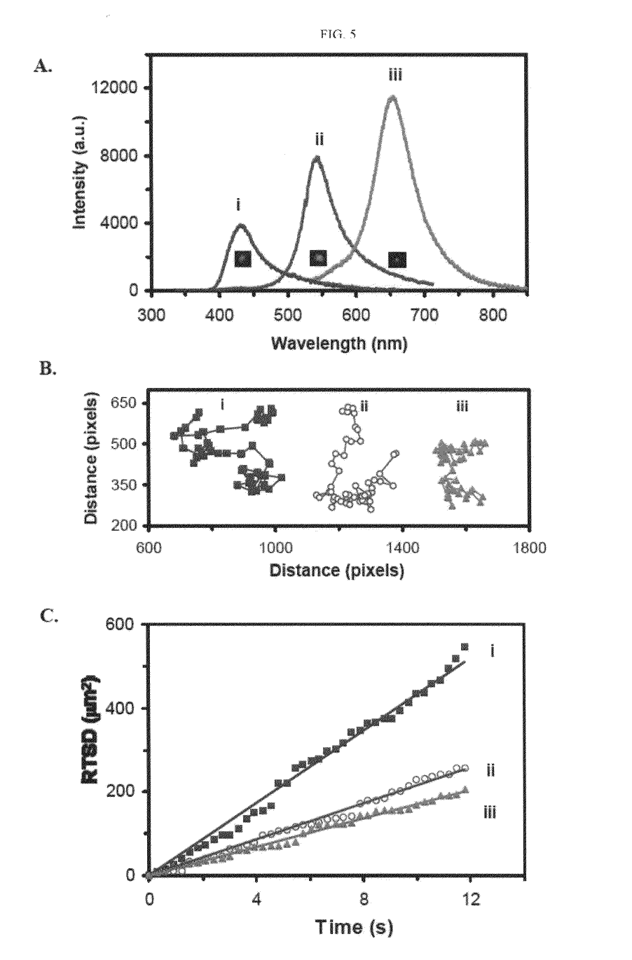

The diffusion of single nanoparticles in solution was characterized and the size-dependence of the nanoparticle diffusion was determined using SNOMS. The size of nanoparticles was determined in solution based on their size-dependent LSPRS (color). The LSPR spectra of three representative colors (blue, green, and red) of individual nanoparticles in FIG. 5A show peak wavelengths at 432, 543, and 655 nm, respectively.

FIG. 5B shows real-time diffusion trajectories of single nanoparticles recorded simultaneously in aqueous solution using a CCD camera with an acquisition rate of 3.3 frames per second (fps). The trajectories show that the smallest nanoparticle (blue) traveled the largest area while the largest nanoparticle (red) diffused in the smallest zone. These results illustrate the dependence of diffusion coefficients on the sizes (radii) of the individual nanoparticles, as described by the Stoke-Einstein equation, D=kT/(6πηa) (Tinoco, I.; Sauer, K.; Wang, J.; Puglisi, J. D., Molecular Motion and Transport Properties. In Physical Chemistry-Principles and Applications in Biological Sciences, Prentice Hall: 2002; pp 274-290.), showing that the diffusion coefficient (D) depends on the viscosity of medium (η) and the radii (a) of solute (nanoparticle). Thus, in order to probe and compare simultaneously multiple nano-environments or multiple molecules in vivo (e.g., intracellular and intra-embryonic fluids) in real-time, it is desirable to use a plurality of nanoparticles having a substantially monodisperse size distribution and to use SNOMS to monitor those nanoparticles in real-time.

To track the diffusion of individual nanoparticles in solution in real-time, RTSD (square of diffusion distance at a given time interval), instead of average (mean) of square-displacement over time was used. Thus, RTSD was used to measure the gradient of viscosity of the medium in real-time as the nanoparticles diffused. This approach can probe the diffusion of single nanoparticles in solution with at least millisecond (ms) temporal resolution in real-time. Plots of RTSD versus diffusion time of the single nanoparticles in FIG. 5C were linear, indicating that single nanoparticles diffuse by simple Brownian motion. The diffusion coefficient (D) of blue, green, and red nanoparticles, calculated by dividing the slope of a linear plot of RTSD versus time by 4 (Note: RTSD=4DΔt), were 10.42×10⁻⁸, 5.23×10⁻⁸, and 3.99×10⁻⁸ cm²/s, respectively.

### Breeding and Monitoring of Zebrafish Embryos:

Wild type adult zebrafish (Aquatic Ecosystems) were bred and maintained according to the methods described in Westerfield, M., The zebrafish book: A Guide for the Laboratory Use of Zebrafish (Danio Rerio*) (http://zfin.org/zf info/zfbook/zfbk.html) ed.; University of Oregon Press Eugene, Oreg., 1993; Vol. Chapters 1 and 2, Chapters 1-4. The embryos were collected at the segmentation stage (21 hpf: 20-25 somites), transferred into a petri dish containing egg water (1.2 mM stock salts in DI water), washed twice with egg water to remove the surrounding debris, and placed directly into a microwell containing 0.2 nM of the Ag nanoparticles from Example 1 in DI water.

### Probing and Imaging Embryos in Real-time:

Zebrafish embryos were selected because they were transparent and developed outside of their mothers. Zebrafish (Danio rerio) is an important vertebrate model system used for an array of studies, such as addressing important questions of developmental biology; see for example Kusumi et al., J. Cell Biology 125, 1251 (1994). Additionally, the embryonic development of zebrafish embryos is completed rapidly, within 120 hours-post-fertilization (hpf), with well-characterized developmental stages, allowing probing of events inside the embryos in vivo in real-time.

The segmentation stage of zebrafish embryos is important in embryonic development during which the foundation and organization of the axial skeleton and the skeletal muscles of the vertebral column are being assembled. (See Stickney, H. L.; Barresi, M. J. F.; Devoto, S. H. Somite Development in Zebrafish. Dev. Dyn. 2000, 219, 287-303.) Current studies show that each developing somite (vertebral segment) contains what will become two main tissue types, sclerotome and myotome, which give rise to the axial skeleton and skeletal muscle for locomotion, respectively. Although it remains unknown how somitogenic timing is regulated and how embryonic morphogenesis and cell migration are controlled (see Luckenbill-edds, L. Am. Zoo. 1997, 37, 213-219), genetic studies have demonstrated that certain signaling proteins exhibit critical functions in determining the organization of embryonic asymmetrical pattern formation and organ placement in development. Notably, diffusion and movements of motor proteins and signaling molecules highly depend on the flow patterns and viscosity gradients of embryonic fluids. Thus, important information can be obtained by imaging flow patterns and viscosity gradients of embryonic fluids in vivo in real-time while monitoring the development of embryos.

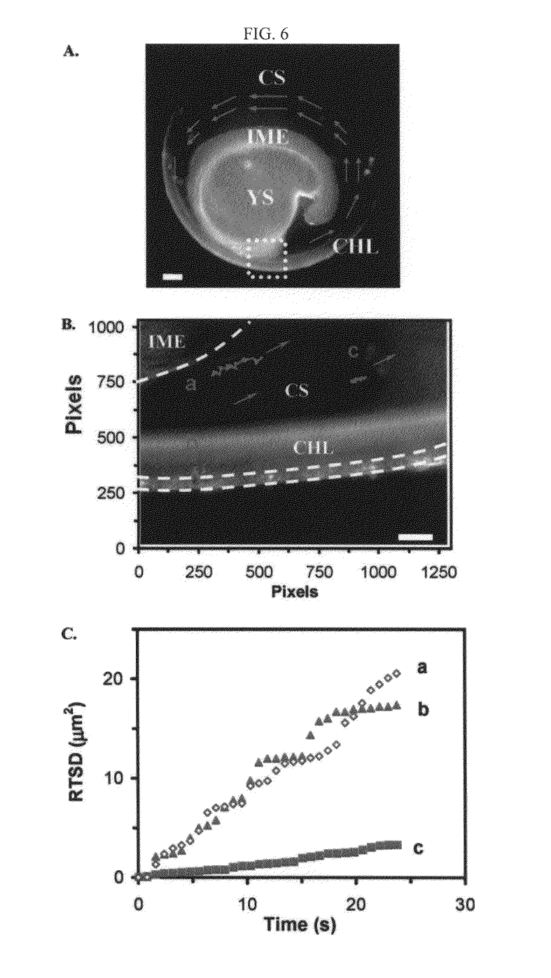

A representative optical image of a segmentation-stage zebrafish embryo in FIG. 6A shows key components of the embryo, including the inner mass of the embryo (IME), the yolk sac (YS), the chorion space (CS), and the chorion layers (CHL) of the embryo. The zoom-in image of the area marked by the dashed square of FIG. 6A shows the interface area of the chorion layers with egg water medium (extra-embryonic), the interface area of the chorion space with the inner mass of embryo, and the chorion space (FIG. 6B).

To this end, the diffusion of three Ag nanoparticles with substantially identical color (green) located in three representative areas of the chorion fluid of segmentation-stage zebrafish embryos were simultaneously imaged and monitored, in order to map the flow patterns and viscosity gradients of embryonic fluids. As described above, a low concentration of Ag nanoparticles (0.2 nM) was used to incubate with segmentation-stage embryos, and the diffusion measurements were completed in minutes. At such a low concentration and short exposure time, the effect of toxicity of Ag nanoparticles on the embryonic development was minimized, as described in Example 3, below. The diffusion trajectories of the substantially identical nanoparticles were significantly different in different locations, as shown in FIG. 6B. Notably, the individual Ag nanoparticles having substantially identical colors (LSPR) have similar sizes (radii), as illustrated in FIG. 2D. The diffusion and trajectory of three green Ag nanoparticles at varying distances from the embryonic surface exhibited counterclockwise flow patterns (FIG. 6B) and a wide range of viscosity gradients of the chorion fluid. In this embodiment, the nanoparticles near the IME showed the maximum linear displacement (FIG. 6B, a), and the nanoparticles in the center of the chorion space were the most restricted (FIG. 6B, c). Overall, the nanoparticles were all moving in counterclockwise direction with flow rates ranging from 0.6 to 1.8 μm/s. Although cilia-driven fluid flows have been observed in the zebrafish pronephros, brain, and kupffer's vesicle (see Kramer-Zucker, A. G.; Olale, F.; Haycraft, C. J.; Yoder, B. K.; Schier, A. F.; Drummond, I. A. Development 2005, 132, 1907-1921), the counterclockwise flow patterns have not previously been reported in zebrafish chorion space.

The nanoparticles near the chorion layers (CHL), at the interface of the intra- and extra-embryo, moved along the membrane and moved in and out of chorion pore canals (CPCs), showing a stepwise plot of RTSD versus diffusion time (FIGS. 6B and 6C: b). As the nanoparticles moved out of chorion pore canals, they exhibited simple Brownian motion with D of 2.2×1 cm²/s. As the nanoparticles diffused into chorion pore canals, their diffusion patterns were restricted (steps in the plot), suggesting that the nanoparticles were trapped in the chorion pore canals, which altered their normal diffusion. Using the plot of RTSD versus time, the entry of individual nanoparticles into chorion pore canals was tracked and it was found that the period of time that individual nanoparticles stayed in the pores ranged from 0.1 to 15 s.

A similar phenomenon was observed for the nanoparticles diffusing at the interface of the chorion space and the inner mass of embryo. FIGS. 6B and 6C: a show a stepwise plot of RTSD versus time, illustrating that the nanoparticles exhibit Brownian diffusion with D of 2.3×10⁻⁹ CM²/s. As the nanoparticles entered the inner mass of embryo, they displayed confined diffusion. Interestingly, the diffusion coefficients of nanoparticles near the inner mass of the embryo was similar to that near the chorion layers, showing similar viscosity. Surprisingly, the nanoparticles near the center of the chorion space displayed Brownian diffusion with the lowest diffusion coefficient of 2.8×10⁻¹⁰ cm²/s (FIGS. 6B and 6C: c), showing the highest viscosity. Using the same approach, similar locations in two other embryos were investigated and similar phenomena were found in that the nanoparticles near the center of the chorion space showed a lower diffusion coefficient, ranging from 2.8×10⁻¹⁰ to 5.0×10⁻⁹ cm²/s, and that a high diffusion coefficient in the chorion space near the inner mass, where high viscosity gradients (about two orders of magnitude higher than that in the chorion space) were observed. It was observed that the viscosity was the highest around the center of chorion space and the lowest in the area of chorion space that were either near the chorion layers or the inner mass of embryos.

The viscosity inside the embryo was about 20 to about 188 times larger than the viscosity of the egg water, suggesting that the chorion layers were not porous enough to establish equilibrium of molecular (nanoparticle) transport. In addition, the slopes of the plots of RTSD versus time, shown in FIG. 6C, were not uniform through the entire measurements, indicating high non-uniformity of the chorion space and emphasizing the importance of probing these nano-environments in real-time.

### Example 3

### An Alternative Embodiment of Synthesis and Characterization of Stable Ag Nanoparticles

This example demonstrates alternative embodiments of the methods for producing silver nanoparticles that are photostable and resist aggregation in solution.

### Synthesis and Characterization of Silver Nanoparticles.

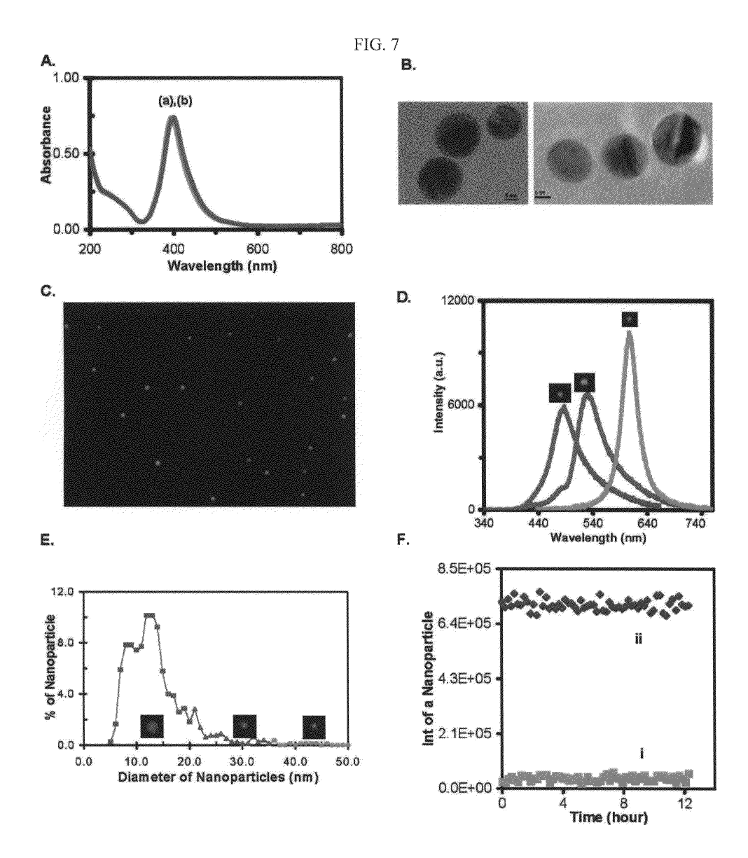

Silver nanoparticles with an average diameter of about 11.6±3.5 nm were synthesized by reducing a 0.1 mM silver perchlorate solution with a freshly prepared ice-cold solution of 3 mM sodium citrate and 10 mM sodium borohydride and stirred overnight; the solution was then filtered through a 0.22 μm filter. The nanoparticles were washed twice with nanopure water using centrifugation to remove the chemicals involved in nanoparticle synthesis, and the nanoparticle pellets were resuspended in nanopure water before incubation with embryos. The washed Ag nanoparticles were very stable (non-aggregated) in nanopure water for months and remained stable in egg water throughout the experiments (120 hpf). The supernatants of the nanoparticle solutions after the second washing were collected for control experiments to study the effect of trace chemicals involved in nanoparticle synthesis on the development of embryos. The concentration, optical properties, and sizes of the nanoparticles were characterized using UV-vis spectroscopy SNOMS, HR-TEM (FEI Tecnai G2 F30 FEG), and DLS (Nicomp 380ZLS particle sizing system); see FIG. 7A to 7F. An electron-multiplying charge-coupled device (EMCCD) or LN back-illuminated CCD camera coupled with a SpectraPro-150 (Roper Scientific) was used in this study. All chemicals were purchased from Sigma and used without further purification or treatment.

The absorption spectra of freshly prepared and washed nanoparticles before and after incubation with egg water for 120 h (FIG. 7A: a and b) show an absorbance of 0.736 at a peak wavelength of 396-400 nm, indicating that the Ag nanoparticles were very stable (not aggregated) in egg water (1.2 mM NaCl). The effect of salt concentration (the positive control experiment) was determined by increasing the NaCl concentration, and it was found that nanoparticles were stable in the presence of NaCl up to 10 mM but begin to aggregate in 100 mM NaCl, showing a red shift in the peak absorbance wavelength (about 2 to 3 nm) and a decrease in absorbance. The size of nanoparticles, measured by DLS, increased from 10.1±2.0 to 24.4±2.7 nm in the presence of 100 mM NaCl. The presence of a sufficiently high concentration of NaCl (100 mM) appeared to reduce the thickness of the electric double-layer on the surface of nanoparticles and decrease the zeta-potential below its critical point, leading to aggregation of nanoparticles.

A representative optical image of single nanoparticles in FIG. 7C illustrates that the majority of nanoparticles were blue, some were green and a few were red. The representative LSPR spectra of single blue, green, and red nanoparticles show peak wavelengths at 488, 532, and 607 nm (FIG. 7D), respectively. The correlation of the color distribution of individual nanoparticles with their size, measured by HR-TEM, showed that the majority (74%) of single nanoparticles, with diameters of 5 to 15 nm, were blue, 23% of single nanoparticles, with diameters of 16 to 30 nm, were green, and a very small fraction (1%) of nanoparticles, with diameters of 31 to 46 nm, were red (FIG. 7E). Thus, the color index of individual nanoparticles can be used as a size index to directly distinguish and determine the sizes of nanoparticles (5 to 46 nm) using SNOMS, even though the sizes of nanoparticles cannot be measured directly due to the optical diffraction limit. The distribution of color and size of the nanoparticles was found to remain unchanged as nanoparticles were incubated in egg water for 120 h, suggesting that the nanoparticles were stable (not aggregated) in egg water at single nanoparticle resolution.

To determine the photostability of Ag nanoparticles, sequential images of single Ag nanoparticles were acquired while those nanoparticles were constantly radiated under a dark-field microscope illuminator (100 W halogen) for 12 h. The illumination power at the sample stage (focal plane of dark-field) was 0.070±0.001 W. Representative plots of scattering intensity of single nanoparticles and background (in the absence of nanoparticles) versus illumination time in FIG. 7F indicate that the scattering intensity of individual nanoparticles remains unchanged over 12 h, showing that single the Ag nanoparticles resist photodecomposition and blinking. Note that the small fluctuations in the scattering intensity from single nanoparticles (FIG. 7F, i) were similar to those observed in the background (FIG. 7F, ii), suggesting that the intensity fluctuations were attributable to the illuminator and the noise level of the CCD camera.

Characterization of Nanoparticles Embedded inside Embryos and Fully Developed Zebrafish.

To characterize the embedded nanoparticles in the tissues of treated zebrafish, living developed zebrafish that had been chronically incubated with a given concentration (0.04 nM) of nanoparticles for 120 hpf since their cleavage (8-cell) stage were selected and carefully rinsed with DI water to remove external nanoparticles. The fixed zebrafish specimens were prepared with 10% buffered formalin via a standard histology protocol of tissue sample preparation; see for example Mohideen et al., Dev. Dyn. 228, 413-423 (2003). Thin-layer microsections (˜5 μm thickness) of tissue samples were prepared by carefully dissecting the tissues of interest (e.g., eye retina, brain, heart, gill arch, tail, and spinal cord) under microscopy using a microtome. The embedded nanoparticles in the tissues were directly characterized using SNOMS.

### Example 4

### Imaging of Transport of Single Nanoparticles in Embryos and Using in Vivo Assays to Probe and Characterize the Biocompatibility and Toxicity of Nanoparticles

This example illustrates the use of silver nanoparticles in an imaging system in one embodiment for studying the transport and biocompatibility of individual silver nanoparticles, made in accordance with Example 1, in and with zebrafish embryos in vivo in real-time.

### Probing Diffusion and Transport of Single Nanoparticles in Cleavage-Stage Embryos.

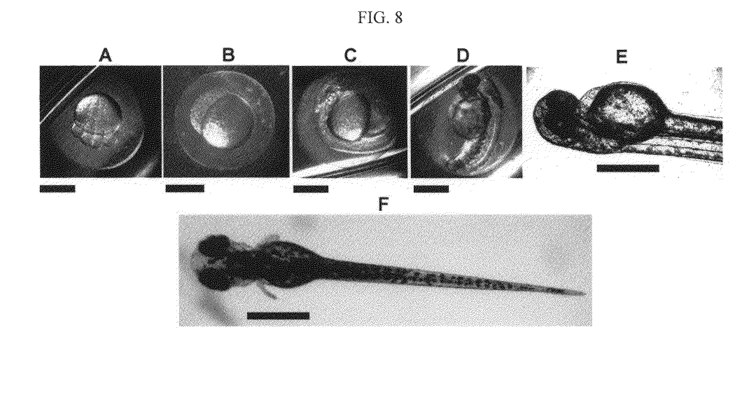

Representative developmental stages of the zebrafish embryos in the 120 hours post-fertilization (hpf) in FIG. 8 are the cleavage-stage embryo (8-64-cell stage), segmentation-stage embryo, hatching-stage embryo, larval-stage embryo, and fully developed zebrafish in the absence of nanoparticles. At the cleavage stage (8-64-cell stage; 0.75-2.25 hpf) (FIGS. 8A and 8B), embryos undergo dramatic changes (e.g., rapid cellular division and distinct fate establishment) to lay the foundation for developing the different parts of organs, and various biochemical and biophysical events (e.g., cell migration and signaling, and embryonic pattern formation). Thus, it is important to understand the diffusion and transport mechanisms among the various parts of the embryo at this particular stage. Cleavage-stage embryos are very sensitive to foreign substances, offering an ultrasensitive in vivo model system to study the biocompatibility and subtle effects of nanoparticles on the embryonic development.

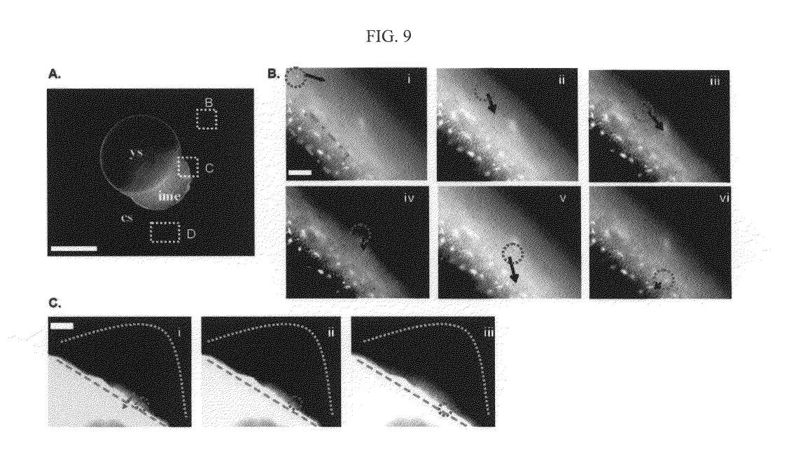

To study the diffusion and transport of single nanoparticles into cleavage-stage embryos, Ag nanoparticles with the embryos were incubated and observed and characterized directly for their transport. It was shown that Ag nanoparticles (blue, green, and red) transported into the chorionic space (CS) via chorion pore canals (CPCs) and entered into the inner mass of the embryo (IME) (FIG. 9). With optical imaging, the chorion pore canals were shown to be approximately 0.5 to 0.7 μm in diameter, with distances between the centers of two nearby chorion pore canals of about 1.5 to 2.5 μm. The chorion pore canals were found to be larger than the nanoparticles, permitting the passive diffusion of individual nanoparticles into the chorionic space of embryos.

To determine the transport mechanism of Ag nanoparticles, two-dimensional mean square displacement (MSD) and diffusion models (e.g., directed, simple, and stationary Brownian diffusion) were utilized to investigate each diffusion trajectory of individual nanoparticles in egg water, entry into embryos, and inside embryos. To follow the diffusion of single nanoparticles inside various parts of embryos in real time, RTSD (diffusion distance at a given time interval), instead of the average (mean) of square displacement over time, was used. This approach allowed probing of the diffusion of single nanoparticles and the viscosity of the different parts of embryonic fluids (e.g., chorionic space, inner mass of the embryo) in real time.

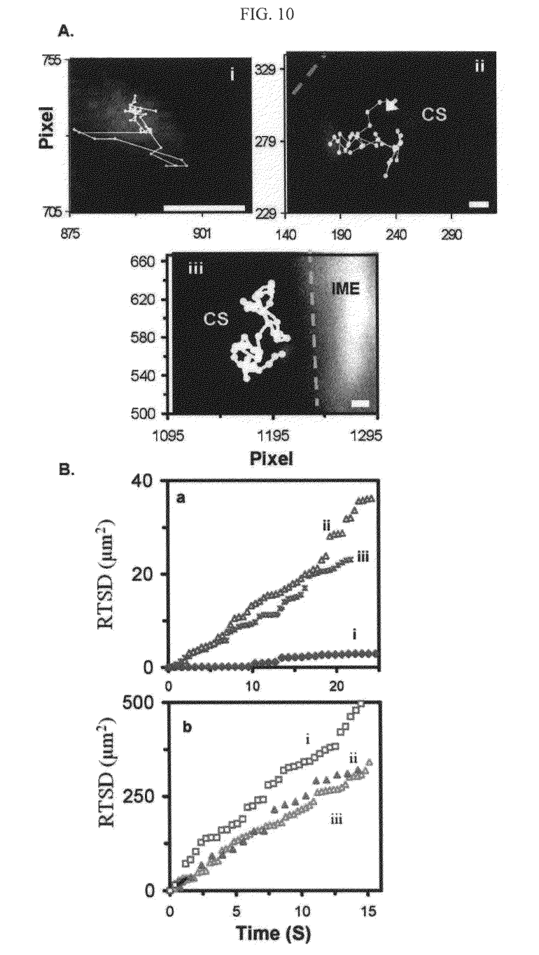

Representative diffusion trajectories of single Ag nanoparticles trapped inside chorion pore canals, in chorionic space, and near the inner mass of the embryo, and analysis of these diffusion trajectories using the RTSD method, are shown in FIG. 10. The results illustrate that single Ag nanoparticles inside the chorionic space (near either the chorion layers or the inner mass of the embryo) exhibit simple Brownian diffusion (not active transport) with diffusion rate (3×10⁻⁹ cm²/s) about 26 times lower than that in egg water (7.7×10⁻⁸ cm²/s), showing that single Ag nanoparticles diffuse into the chorionic space via passive diffusion and that the viscosity of chorionic space is about 26 times higher than that of egg water.

As nanoparticles entered the chorion layers and inner mass of the embryo, their diffusion patterns were restricted (FIG. 10B, a-i, steps in a-ii and a-iii), suggesting that the nanoparticles docked into the chorion pore canals, which limited their normal diffusion. By tracking the entry of individual nanoparticles into chorion pore canals, the period of time that individual nanoparticles stayed in the pores was found to range from 0.1 to 15 s.

The diffusion coefficients of blue, green, and red nanoparticles in egg water (FIG. 10B, b) were measured to determine the possible variation of diffusion coefficients due to the different sizes (radii) of single nanoparticles, which showed simple Brownian diffusion with D=8.4×10⁻⁸, 6.0×10⁻⁸, and 5.5×10⁻⁸cm²/s, respectively. The diffusion coefficients of the given color (radius) nanoparticles in embryos were studied and compared with those in egg water, showing that the various diffusion coefficients observed in three different parts of embryos (FIG. 10B, a) were indeed attributable to the viscosity gradient inside the embryos, but not the different radii of individual nanoparticles.

Characterization of Transport and Embedded Nanoparticles.

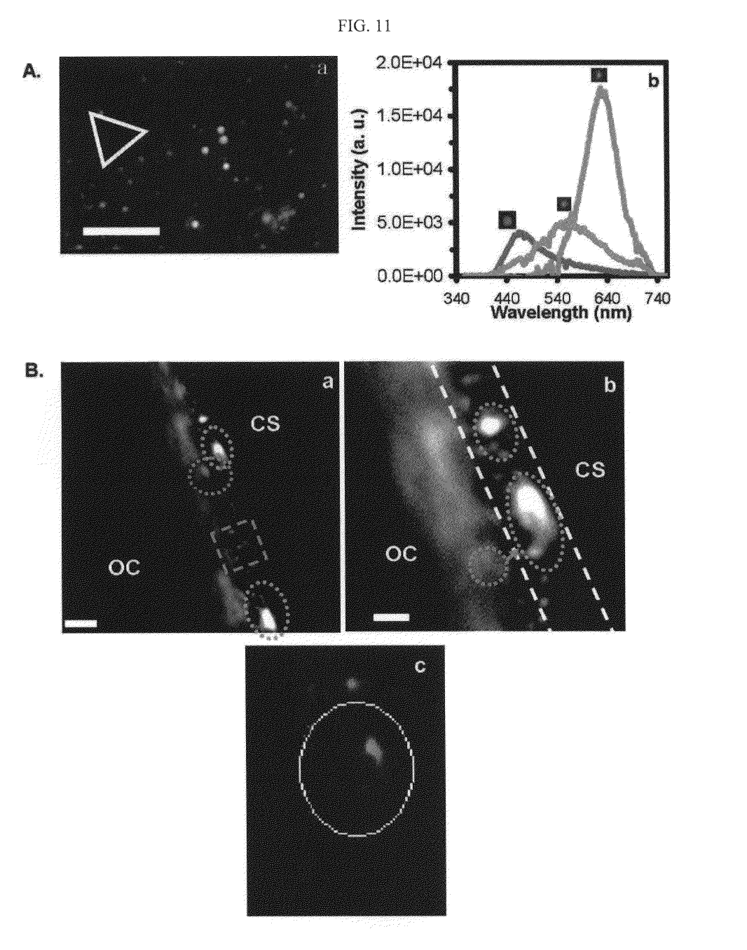

Images of the transport of single nanoparticles into the chorionic space were recorded using dark-field SNOMS equipped with a color camera, instead of a CCD, showing that nanoparticles of multiple colors transport into the chorionic space (FIG. 11A). Note that single Ag nanoparticles exhibit colors (LSPR), which depend on the size and shape of the nanoparticle and the surrounding environment. This feature was used to distinguish single Ag nanoparticles from tissue debris or vesicle-like particles in embryos, which did not exhibit surface plasmon and hence appeared white under dark-field microscopy. The majority of nanoparticles were found to be transported into the chorionic space, and some of them overlapped with chorion pore canals (FIG. 11A: a). The representative LSPR spectra (colors) of individual nanoparticles inside the chorionic space (FIG. 11A: b) showed peak wavelengths similar to those observed in egg water. The results indicate that the majority of nanoparticles remained non-aggregated inside the embryos.

Although the majority of single nanoparticles freely diffused into embryos and remain non-aggregated, some single nanoparticles stayed in chorion pore canals for an extended period of time. These trapped nanoparticles served as nucleation sites and aggregate with incoming nanoparticles to form larger particles (dark-red nanoparticles, FIG. 11B), clogging chorion pore canals and affecting the embryo's transport. Note that embryos at this developmental stage are free of pigmentation.

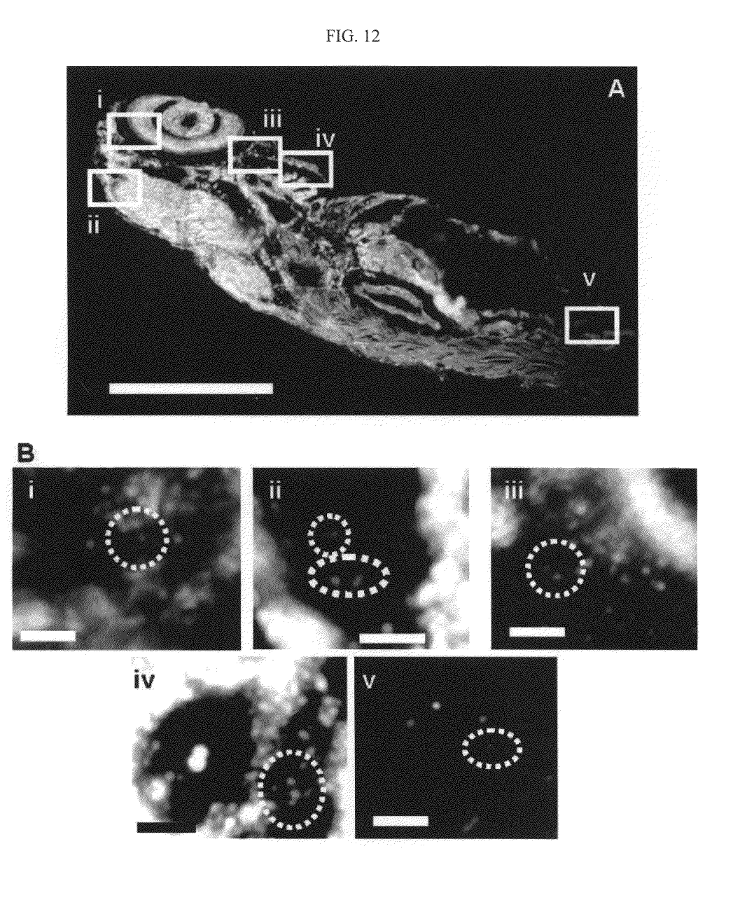

As the cleavage-stage (8-cell) embryos chronically treated with lower concentrations of Ag nanoparticles (<0.08 nM) completed their embryonic development at 120 hpf, Ag nanoparticles embedded in fully developed zebrafish using SNOMS were characterized. It was found that these Ag nanoparticles embedded in multiple organs (retina, brain, heart, gill arches, and tail) of normally developed zebrafish (FIG. 12), demonstrating that Ag nanoparticles are biocompatible with embryos at lower concentrations (<0.08 nM). The LSPR spectra of these embedded nanoparticles are similar to those shown in FIG. 11A: b. Blank control experiments were also conducted by imaging 120-hpf zebrafish that developed in the absence of nanoparticles and did not observe the signature LSPR spectra (color) of Ag nanoparticles in these fully developed zebrafish.

### In Vivo Assay for Probing Dose-Dependent Biocompatibility and Toxicity of Nanoparticles.

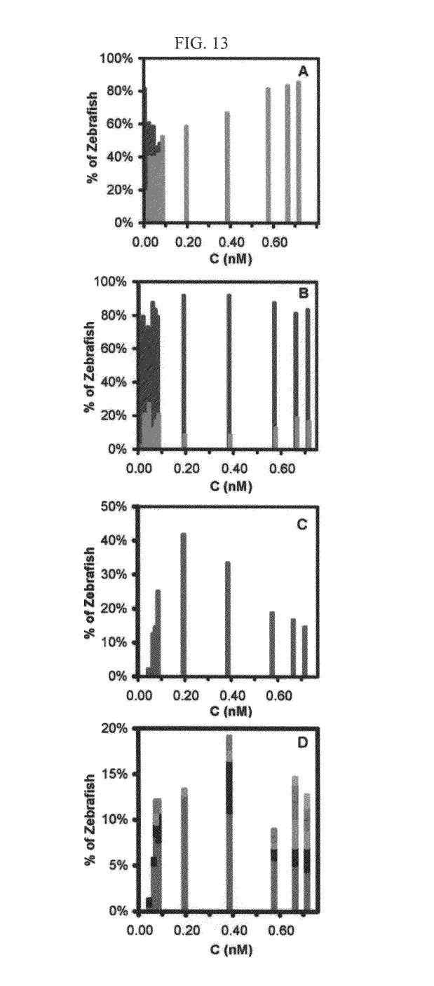

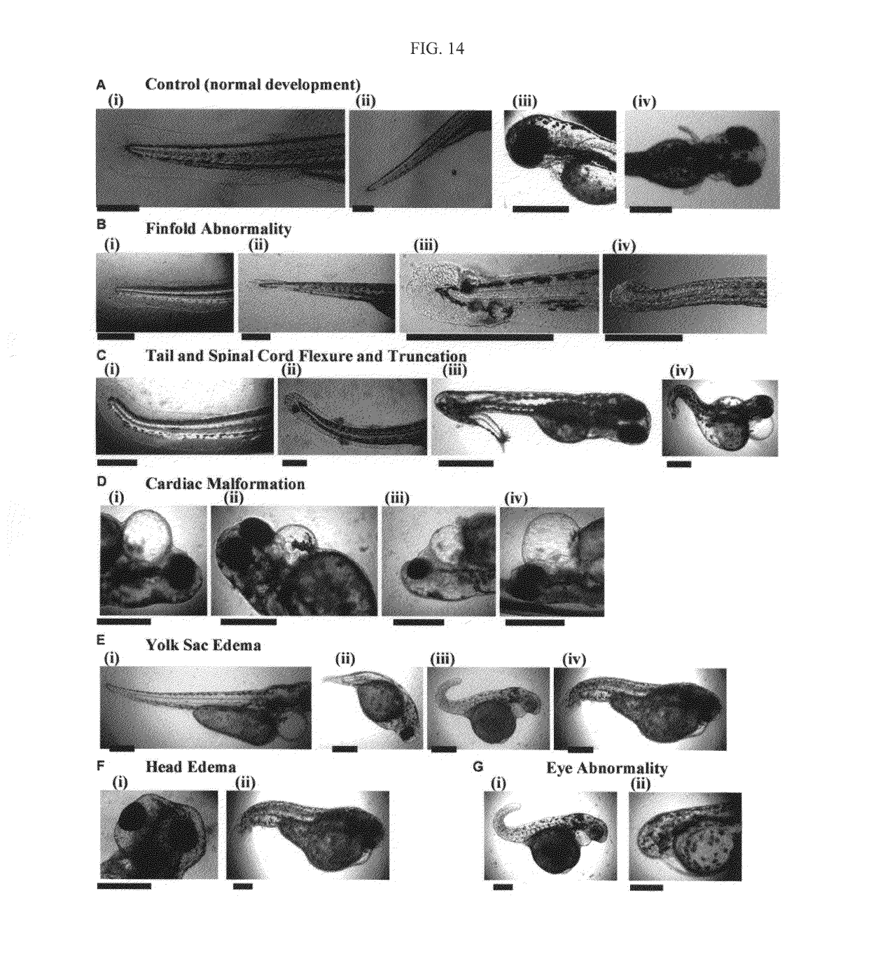

To determine the effect of different doses of Ag nanoparticles on embryonic development, the cleavage-stage (8-cell) embryos were treated chronically with various concentrations of Ag nanoparticles (0-0.71 nM) and carefully monitored and characterized at the vital developmental stages (24, 48, 72, 96, and 120 hpf). The results in FIGS. 13 and 14 show that biocompatibility and toxicity of Ag nanoparticles and the types of abnormalities in treated zebrafish were both highly dependent on the dose of Ag nanoparticles. In the presence of lower concentrations (<0.08 nM) of nanoparticles, the percentage of normally developed zebrafish observed was higher than that of dead and deformed zebrafish. Note that both normal and deformed zebrafish developed from the cleavage-stage (8-cell) embryos that had been simultaneously incubated with the same nanoparticle solution.

As nanoparticle concentration increased, the number of normally developed zebrafish decreased, while the number of dead zebrafish increased (FIG. 13A). As nanoparticle concentration increased beyond 0.19 nM, only dead and deformed zebrafish were observed, showing a critical concentration of Ag nanoparticles in the development of zebrafish embryos (FIG. 13). The blank (negative) control experiments, conducted by replacing nanoparticles with the supernatant resulting from washing Ag nanoparticles, showed that the survival rate of zebrafish was independent of the dose of supernatant (FIG. 13B), demonstrating that residual chemicals from nanoparticle synthesis were not responsible for the deformation and death of zebrafish, but rather the nanoparticles that were used to treat the zebrafish embryos (FIG. 13A).

The number of deformed zebrafish increased to its maximum as nanoparticle concentration increased to 0.19 nM, and then decreased as nanoparticle concentration increased from 0.19 to 0.71 nM (FIG. 13C) because the number of dead zebrafish increased. Interestingly, the types of viable deformities exhibit a high dependence on the nanoparticle concentration (FIG. 13D). For example, finfold abnormality and tail/spinal cord flexure and truncation were observed in zebrafish treated with all tested nanoparticle concentrations (0.04-0.71 nM), with the highest occurrences at 0.19 and 0.38 nM, respectively. Cardiac malformation and yolk sac edema were observed in zebrafish treated with slightly higher nanoparticle concentrations (0.07-0.71 nM), with the highest occurrences at 0.66 nM. In contrast, head edema and eye deformity were observed only with the higher concentrations of nanoparticles, 0.44-0.71 and 0.66-0.71 nM, respectively. Among all types of observed deformities, finfold abnormality occurred at the highest rate, followed by tail and spinal cord flexure and truncation, and then cardiac malformation and yolk sac edema, and finally head edema and eye abnormality, which were rarely observed deformations of zebrafish and quickly led to zebrafish death.

Representative zebrafish deformations induced by nanoparticles are illustrated in FIGS. 14B-14G. In comparison with the normally developed zebrafish shown in FIG. 14A, the deformations in zebrafish that developed from the 8-cell embryos treated chronically by Ag nanoparticles showed characteristics of finfold abnormality (FIG. 14B), tail and spinal cord flexure and truncation (FIG. 14C), cardiac malformation (FIG. 14D), yolk sac edema (FIG. 14E), head edema (FIG. 14F), and eye abnormality (FIG. 14G). Interestingly, multiple deformities could occur in a single zebrafish at the higher nanoparticle concentrations (>0.38 nM). For example, in yolk sac edema zebrafish, tail/spinal cord flexure, finfold abnormality, and cardiac malformation (FIGS. 14C-iv and E-ii), head edema (FIGS. 14E-ii, E-iv, and F-ii), and eye abnormality (FIG. 14G-i) were observed.

To determine possible targets for further genomic and proteomic studies and evaluate the toxicity of Ag nanoparticles against well-studied toxic chemicals, such as cadmium, dichloroacetic acid (DCA), 2,3,7,8-tetrachlorodibenzo-p-dioxin (TCDD), and ethanol, the characteristics of deformation of zebrafish induced by Ag nanoparticles were compared with those generated by well-known toxic chemicals. The observed finfold abnormality and tail/spinal cord flexure and truncation induced by Ag nanoparticles (FIGS. 14B and 14C) were found to be similar to those observed in zebrafish treated with DCA and cadmium, indicating possible common targets of malformation during development. The observed cardiac malformation and yolk sac edema in this study (FIGS. 14D and 14E) were also similar to those observed in zebrafish treated with DCA and cadmium. The shrunken ventricular myocardium observed in cardiac malformed zebrafish induced by nanoparticles (FIG. 14D) is similar to the observation in zebrafish treated with TCDD. Head edema and eye abnormality (FIGS. 14F and 14G-i) have also been found to result in zebrafish treated with cadmium. For cadmium related studies, see for examples Hallare et al., J. Therm. Bio. 30, 7-17 (2005).

For the purposes of this disclosure, and unless otherwise specified, “a” or “an” means “one or more.” All patents, applications, references, and publications cited herein are incorporated by reference in their entirety to the same extent as if they were individually incorporated by reference.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like includes the number recited and refers to ranges which can be subsequently broken down into subranges as discussed above. Finally, as will be understood by one skilled in the art, a range includes each individual member.

What is claimed is:

1. A method for forming a stabilized nanoparticle dispersion, the method comprising:
providing nanoparticles having a surface zeta potential;
increasing the absolute value of the surface zeta potential of the nanoparticles to provide stabilized nanoparticles; and
dispersing the stabilized nanoparticles in an aqueous dispersion medium, wherein the dispersed nanoparticles exhibit improved stability against aggregation in the aqueous dispersion medium.

2. The method of claim 1, wherein the absolute value of the zeta potential is increased by at least about 50%.

3. The method of claim 1, wherein the nanoparticles are capable of remaining substantially non-aggregated in the aqueous dispersion medium for at least two months.

4. The method of claim 1, wherein the absorption spectra of the nanoparticles in the aqueous dispersion medium are capable of remaining substantially unchanged for at least one month.

5. The method of claim 1, wherein the nanoparticles do not exhibit photobleaching or blinking for a period of at least one hour.

6. The method of claim 1, wherein the nanoparticles comprise a noble metal.

7. The method of claim 6, wherein the nanoparticles are capable of remaining substantially non-aggregated in the aqueous dispersion medium for at least two months.

8. The method of claim 6, wherein the absorption spectra of the nanoparticles in the aqueous dispersion medium are capable of remaining substantially unchanged for at least one month.

9. The method of claim 6, wherein the nanoparticles do no exhibit photobleaching or blinking for a period of at least one hour.

10. The method of claim 6, wherein the nanoparticles are silver nanoparticles.

11. The method of claim 10, wherein the stabilized silver nanoparticles have a surface zeta potential of −30 mV or lower.

12. The method of claim 1, wherein increasing the absolute value of the surface zeta potential of the nanoparticles comprises washing the nanoparticles with a washing agent that increases the thickness of the electrical double layer around the nanoparticles.

13. The method of claim 12, wherein washing the nanoparticles with the washing agent is carried out at least twice.

14. The method of claim 12, wherein the washing agent is deinoized water.

15. An aqueous solution comprising dispersed, non-aggregated nanoparticles, wherein the solution is substantially free of steric stabilizing agents and further wherein the nanoparticles are capable of remaining substantially non-aggregated in the aqueous solution for at least one month.

16. The solution of claim 15, wherein the absorption spectra of the nanoparticles in the aqueous solution are capable of remaining substantially unchanged for at least one month.

17. The solution of claim 15, wherein the nanoparticles do no exhibit photobleaching or blinking for a period of at least twenty four hours.

18. The solution of claim 15, wherein individual nanoparticles do not undergo photodecomposition or exhibit blinking, for at least about 24 hours.

19. The solution of claim 15, wherein the nanoparticles comprise a noble metal.

20. The solution of claim 19, wherein the nanoparticles are capable of remaining substantially non-aggregated in the aqueous solution for at least one month.

21. The solution of claim 19, wherein the nanoparticles are silver nanoparticles.

22. The solution of claim 21, wherein the stabilized silver nanoparticles have a surface zeta potential of −30 mV or lower.

23. A method for imaging nanoparticles in a biological organism in vivo, comprising:
exposing the biological organism to a plurality of nanoparticles, wherein the nanoparticles diffuse into the biological organism;
simultaneously imaging a plurality of individual nanoparticles within a biological organism in vivo in real-time by detecting light scattered by the nanoparticles, wherein the color of the scattered light is nanoparticle size-dependent.

24. The method according to claim 23, wherein the nanoparticles are present in multiple environments in or around the biological organism, such that the multiple environments of the biological organism are probed simultaneously by imaging individual nanoparticles simultaneously in real-time.

25. The method according to claim 23, wherein imaging the plurality of individual nanoparticles comprises using dark-field single nanoparticle optical microscopy and spectroscopy (“SNOMS”) to determine the color of the light scattered from individual nanoparticles, the method further comprising correlating the color of the scattered light to the size of the individual nanoparticles, whereby imaging of the individual nanoparticles at nanometer-scale resolution is achieved.

26. The method according to claim 25, The method according to claim 23, wherein the biological organism is a vertebrate biological organism.

27. The method according to claim 23, wherein the biological organism comprises a tissue.

28. The method according to claim 23, wherein individual nanoparticles are imaged at single-nanoparticle resolution.

29. The method according to claim 23, wherein the imaging is carried out using dark-field optical microscopy and spectroscopy via direct visualization of the localized surface plasmon resonance of individual nanoparticles.

30. The method according to claim 23, wherein the plurality of individual nanoparticles have a substantially monodisperse size distribution and multiple environments of the biological organism are probed simultaneously by imaging the nanoparticles at single-nanoparticle resolution in real-time.

31. The method according to claim 23, wherein the plurality of individual nanoparticles have a substantially monodisperse size distribution and are located on a surface of, at an interface of, or within the biological organism, and further wherein multiple environments of the biological organism are probed simultaneously by imaging the individual nanoparticles in real-time.

32. The method according to claim 23, wherein the nanoparticles comprise a noble metal.

33. The method according to claim 32, wherein the nanoparticles are silver or gold nanoparticles.

34. The method according to claim 23, wherein the nanoparticles are produced according to method of claim 1.

35. The method according to claim 23, wherein imaging the individual nanoparticles provides information about the fluid viscosity of multiple environments within the biological organism.

36. The method according to claim 23, wherein imaging the nanoparticles provides information about the fluid flow of multiple environments within the biological organism.

37. The method according to claim 23, wherein imaging the nanoparticles provides information about transport dynamics and mechanisms in the biological organism.

38. The method according to claim 23, wherein imaging has single nanoparticle resolution at nanometer-scale and further wherein imaging the nanoparticles provides information about the biocompatibility of the nanoparticles with the biological organism.

39. The method according to claim 23, wherein the biological organism comprises an embryo.

40. The method according to claim 39, wherein the embryo is a zebrafish embryo.

41. The method according to claim 42, wherein the diffusion of the nanoparticles through the chorion pore canals of a living zebrafish embryo is imaged in real-time.

42. The method according to claim 23, wherein the imaging is performed continuously for a period of at least 1 hour.

43. The method according to claim 23, wherein the imaging is performed continuously for a period of at least 24 hours.

44. The method according to claim 23, wherein the average diameter of the nanoparticles is at least about 2 nm.

45. A method for determining the effect of nanoparticles on one or more living biological organisms, the method comprising:
exposing the one or more living biological organisms to a plurality of nanoparticles and monitoring the morphology or development of the one or more living biological organisms to determine the biocompatibility or toxicity of the nanoparticles.

46. The method according to claim 45, wherein the one or more living biological organisms are zebrafish embryos.

47. The method according to claim 46, wherein the zebrafish embryos are exposed to nanoparticles at different nanoparticle concentrations and exposure to at least one of the nanoparticle concentrations results in a physical abnormality in, or the death of, one or more of the zebrafish embryos, whereby the concentration dependent biocompatibility of the nanoparticles is determined.

48. The method according to claim 46, wherein the zebrafish embryos are exposed to the nanoparticles for different exposure times and exposure for at least one of the exposure times results in a physical abnormality in, or the death of, one or more of the zebrafish embryos, whereby the exposure time-dependent biocompatibility or toxicity of the nanoparticles is determined.

49. The method of claim 46, wherein exposing the one or more zebrafish embryos to the nanoparticles results in a physical abnormality in one or more of the zebrafish embryos, the method further comprising imaging at least one of the location, amount or size of the nanoparticles in an abnormal zebrafish embryo to determine cause of the abnormality.

50. The method of claim 49, wherein the nanoparticles are produced according to the method of claim 1.

51. A method of transporting nanoparticles into an embryo, the method comprising exposing the embryo to a solution comprising a plurality of nanoparticles, wherein one or more nanoparticles passively diffuse into the embryo.

52. The method of claim 51, wherein the embryo is a zebrafish embryo.

53. A method for imaging, comprising:
imaging a plurality of individual nanoparticles in an aqueous medium or on a substrate by dark-field single nanoparticle optical microscopy and spectroscopy (“SNOMS”) by detecting light scattered by the nanoparticles in real-time.

54. A method of making silver nanoparticles, comprising:
adding at least one silver-containing compound into a solution comprising at least two reducing agents dissolved in water at a temperature of no greater than about 5° C. with constant stirring; and
continuing to stir the solution for a period of at least 12 hours;
wherein the reducing agents reduce the silver-containing compound to form the silver nanoparticles.

55. The method of claim 54, wherein the first of the two reducing agents comprises sodium citrate, the second of the two reducing agents comprises sodium borohydride, and the ratio of sodium citrate to sodium borohydride in the solution is about 1:9 to about 1:11.

56. The method of claim 54, wherein the temperature of the solution is about 0° C. during the formation of the silver nanoparticles.

57. The method of claim 54, further comprising isolating the nanoparticles by filtering the solution through a filter having a mesh size no greater than 2 um and washing the nanoparticles with deionized water.

58. A kit comprising nanoparticles produced according to claim 1 or a solution of nanoparticles according to claim 15.
